(19) United States
(12) Patent Application Publication
(10) Pub. No.: US 2017/0298449 A1
(43) Pub. Date: Oct. 19, 2017

(54) GENE EXPRESSION SIGNATURES FOR DETECTION OF UNDERLYING PHILADELPHIA CHROMOSOME-LIKE (PH-LIKE) EVENTS AND THERAPEUTIC TARGETING IN LEUKEMIA

(71) Applicants: STC.UNM, Albuquerque, NM (US); ST. JUDE CHILDREN'S RESEARCH HOSPITAL, Memphis, TN (US); THE CHILDREN'S HOSPITAL OF PHILADELPHIA ON BEHALF OF CHILDREN'S ONCOLOGY GROUP, Philadelphia, PA (US)

(72) Inventors: Cheryl L. Willman, Albuquerque, NM (US); Stephen P. Hunger, Greenwood Village, CO (US); Charles Mullighan, Memphis, TN (US); I-Ming Chen, Albuquerque, NM (US); Kathryn G. Roberts, Memphis, TN (US); Huining Kang, Albuquerque, NM (US); Richard C. Harvey, Placitas, NM (US)

(21) Appl. No.: 15/638,926

(22) Filed: Jun. 30, 2017

Related U.S. Application Data
(62) Division of application No. 14/364,182, filed on Jun. 10, 2014.
(60) Provisional application No. 61/569,507, filed on Dec. 12, 2011.

(51) Int. Cl. C12Q 1/68

(52) U.S. Cl. CPC C12Q 1/6886; C12Q 2600/106; C12Q 2600/158

(57) ABSTRACT

The invention provides arrays, systems, devices, methods, computer-readable media and kits that enable expression-based classification of B-precursor acute lymphoblastic leukemia (ALL) as being either responsive or non-responsive to tyrosine kinase inhibitor mono or co-therapy.

This application claims priority from U.S. Provisional Application Ser. No. 61/569,507, filed Dec. 12, 2011 and entitled “Gene Expression Signatures for Detection of Underlying Tyrosine Kinase Mutations and Therapeutic Targeting in Leukemia”. The complete contents of this provisional patent application are hereby incorporated by reference.

### RELATED APPLICATIONS AND GRANT SUPPORT

This invention was supported by grant U01 CA114762, U01 CA157937, U01CA98543, IRC2 CA148529 and the National Cancer Institute-funded TARGET (Therapeutically Applicable Research to Generate Effective Treatments) Project on High-Risk Acute Lymphoblastic Leukemia (ALL) (http://target.cancer.gov/) from the National Cancer Institute. Consequently, the government retains rights in the invention.

### BACKGROUND OF INVENTION

Gene expression patterns have been used for several decades to distinguish tissue types, cellular origins, stages of development, and pathogenetic changes in normal and diseased cells. Historically, this has been most commonly practiced in clinical diagnostic laboratories using antibodies to gene products to detect their expression levels and/or subcellular localization. The antibodies may be tagged with detectable markers and then quantified either by light or fluorescence microscopy, flow cytometry, or other comparable techniques. Most commonly, such diagnostic approaches involve only a few gene products in any given sample (alone or in combination) and are limited by the specificity of the antibodies, the expression levels of the proteins, and their accessibility in the cells of interest.

With the advent of improved molecular biological and comprehensive genomic analysis methods, this same concept has now been extended to the analysis of cellular RNA or DNA in the cells of interest, rather than just the resulting protein products. When combined with target amplification techniques such as polymerase chain reaction (PCR), the sensitivity of these methods permits the detection of fewer than ten molecules of a particular analyte in the specimen being tested. Recent technological advances and automated genomic platforms, including gene expression arrays,¹ also now permit the simultaneous interrogation of tens of thousands of gene targets encompassing the entire human genome in a single cell or tissue.

Application of these new methods to human tissue samples has revealed that distinctive patterns of gene expression, often referred to as “gene expression signatures,” are associated with specific phenotypes. In cancer cells, many of these perturbed or altered gene expression signatures have been shown to result from underlying chromosomal rearrangements or translocations, mutations in specific genes that affect their expression, epigenetic changes in the genome, and other cancer-associated and cancer-promoting genetic and epigenetic abnormalities. Such signatures are often thus of use in the clinical setting for diagnosis, determination of outcome (prognosis), prediction of response to therapy, and targeting of patients to specific therapeutic interventions.¹ Such gene expression signatures have also led to the discovery by our group and others of previously unknown recurrent genetic abnormalities in cancer cells (such as IGH@-CRLF2 and P2RY8-CRLF2).²⁻⁴

This invention reports a specific and robust gene expression signature, based on the combinatorial and quantitative expression of a limited set of human genes, which can be used in the clinical diagnostic laboratory setting to screen and prospectively identify those patients diagnosed with B-precursor cell acute lymphoblastic leukemia (ALL) who share a common gene expression signature which results from a highly heterogeneous spectrum of mutations and cryptic translocations involving genes encoding tyrosine kinases.⁵⁻¹¹ As such patients have an exceedingly poor outcome when treated with standard chemotherapy for ALL¹⁻⁸ and will likely benefit from next generation therapies incorporating newer agents, particularly tyrosine kinase inhibitors (TKIs), their prospective identification is clinically important. Thus, this invention enables the screening and prospective identification of a defined subset of ALL patients to facilitate therapeutic targeting.

The classic Philadelphia (Ph) chromosome translocation, or t(9;22)(q34;q11), a hallmark of Chronic Myelogenous Leukemia (CML) and other forms of acute leukemia (particularly ALL), results in a novel chimeric gene and protein which fuses the BCR gene on chromosome 22 with the gene encoding the Abelson tyrosine kinase (ABL1) on chromosome 9. The resulting BCR-ABL1 fusion transcript and protein is a constitutively activated tyrosine kinase which activates various signaling pathways to promote leukemic transformation in hematopoietic stem cells. Targeted inhibition of this activated ABL tyrosine kinase with first generation tyrosine kinase inhibitors (TKIs) such as Imatinib® or Gleevac®, as well as next generation TKIs, has revolutionized the therapy of Ph-positive leukemias, leading to dramatic improvements in patient outcome.¹²

Our group of inventors,^{5,6,8} and subsequently another team of investigators,¹³ first discovered and reported a series of highly related gene expression signatures variously referred to as “cluster group R8,” “Philadelphia Chromosome (Ph)-like,” “Ph-like,” “BCR-ABL1-like,” or an “activated tyrosine kinase gene expression signature,” that defined a distinct subset of patients with ALL who also had an extremely poor outcome when treated on standard chemotherapeutic regimens. Our group first discovered this unique signature when we applied hierarchical clustering and other novel clustering methods to a gene expression dataset derived from the leukemic cells of a cohort of 207 children with high risk ALL who had been accrued to a national clinical trial (P9906) conducted by the Children's Oncology Group (COG) (using the Affymetrix U133 Plus 2.0 array platform containing complete coverage of the human genome plus 6,500 additional genes for analysis of over 47,000 human mRNA transcripts).^{5,6} With this approach, we identified a novel and statistically robust cluster of patients with an exceedingly poor clinical outcome, which we first termed “cluster group R8.”^{5,6} The gene expression signature for ALL patients in cluster group 8, and several of the outlier genes whose high or low expression defined this cluster group,^{5,6} were found to be highly similar to those seen in ALL patients with the classic Philadelphia (Ph) chromosomal translocation.^{12,14} Yet, none of the leukemic cells in this novel “cluster group 8” or “Ph-like” patient group, or in the full cohort of 207 high risk ALL patients examined, contained the classic Ph chromosome translocation or the pathognomonic BCR-ABL1 fusion transcript. In a parallel approach, using a different gene expression analysis method (termed “gene set enrichment”) on the same gene expression data set originally derived in our laboratories, we further demonstrated that children with a “Philadelphia chromosome-like” or “BCR-ABL1-like” gene expression signature had a very poor outcome and frequent deletion of the IKAROS or IKZFI transcription factor regulating B cell development.⁸

Given that this distinct group of ALL cases had a gene expression signature (referred to hereafter as a “Ph-like” gene expression signature) similar to classic Philadelphia chromosome-positive ALL cases but lacked this specific translocation and the BCR-ABL1 fusion gene, we hypothesized that the unique subset of Ph-like ALL patients might have leukemia-promoting mutations or translocations involving one or more genes encoding the other 90 members of the tyrosine kinase human gene family. Over the past two years, under the auspices of the NCI TARGET project (http://target.cancer.gov), our group has employed traditional Sanger sequencing methods for targeted gene resequencing as well as next generation sequencing methods (exon sequencing, whole genome sequencing, and transcriptomic or RNA sequencing) in this and other ALL patient cohorts to identify the underlying genetic mutations in this unique group of Ph-like ALL patients.^{7,9-11} Strikingly, our group has determined that ALL patients with a Ph-like gene expression signature have a highly heterogeneous spectrum of novel mutations and cryptic translocations involving several genes encoding tyrosine kinases in the human genome, including ABL1 itself, the JAK family of tyrosine kinases, the PDGF receptor tyrosine kinase (PDGFR), the IL-7 receptor (IL7R) regulating B cell development, the erythropoietin receptor (EPOR), and genes regulating JAK kinase signaling pathways (LNK).^{7,9-11} As these discovery efforts are ongoing, novel fusions and genetic mutations continue to be identified in this group of patients. To date, we have determined that approximately 50% of ALL patients with a Ph-like gene expression signature in our patient cohorts have genomic rearrangements of CRLF2 (a homologue of the type I cytokine receptor family common gamma signaling chain that heterodimerizes with the IL7R alpha chain to regulate hematopoietic cell development)²⁻⁴ as well as activating point mutations of the JAK family of tyrosine kinases (JAK1/JAK2/JAK3).^{7,9-11} Of the 15 ALL cases with a Ph-like gene expression signature that have undergone transcriptomic sequencing to date (12 selected from the R8 cluster group and 3 cases with this signature derived from the full cohort),^{5,6,8} each case was shown to contain either a cryptic translocation involving a tyrosine kinase (either STRN3-JAK2, EBF1-PDGFRB, NUP214-ABL1, IGH@-EPOR, BCR-JAK2, PAX5-JAK2, ETV6-ABL1, RCSD1-ABL1, or RANBP2-ABL1) or a mutation in IL7R and/or a gene (SH2B3 or LNK) regulating JAK signaling pathways.¹⁰ Importantly, all patients in the original R8 cluster group have been determined to have one of these novel kinase mutations;^{5,6,10} thus the gene expression signature and outlier genes defining this cluster group of ALL patients is particularly robust.

As the treatment of Philadelphia chromosome-positive leukemia patients with tyrosine kinase inhibitors (TKIs) targeting the activated ABL1 kinase, alone or in combination with other chemotherapy, has resulted in dramatic improvements in overall survival,¹² we have hypothesized that ALL patients with a “Ph-like” gene expression signature and a spectrum of mutations involving other tyrosine kinases will similarly achieve improved clinical outcomes when treated with regimens employing TKIs or other targeted agents. Our recent in vitro and in vivo studies using established cell lines, primary Ph-like ALL patient samples, and ALL xenograft models have provided confirmatory data by demonstrating significant growth inhibition of Ph-like ALL cells following exposure to TKIs and other targeted agents.^{9,10,12,15} From our body of work completed to date,¹⁻¹¹ and additional unpublished data, we estimate that Ph-like ALL comprises approximately 10% of pediatric ALL patients considered standard risk, 15-20% of pediatric ALL patients considered high risk, and 35-40% of the ALL cases occurring in adolescents and young adults. Given the relatively high frequency of this gene expression signature and the poor outcome of these patients on standard treatment regimens, it is important to develop a diagnostic screening method to prospectively identify Ph-like ALL cases so that they can be targeted to more effective treatment regimens.

In this invention, we have developed a robust gene expression signature, based on the combinatorial and quantitative expression of a limited number of human genes, which can be used in the clinical diagnostic laboratory setting to screen and prospectively identify Ph-like ALL patients. Since the provisional patent filing, we have further adapted this signature and predictive algorithm, initially derived from gene expression arrays, to a more limited diagnostic gene set which can be measured using quantitative RT-PCR on robust clinical diagnostic platforms. This signature identifies those patients diagnosed with B-precursor cell ALL who share a common gene expression signature which results from a highly heterogeneous spectrum of mutations and cryptic translocations involving genes encoding tyrosine kinases.⁵⁻¹¹ The signature was created by training on ALL cases with known kinase mutations, including: 1) activating mutations of tyrosine kinases (JAK1, JAK2, and IL7R); 2) genes whose loss of function mutations promote activated tyrosine kinase signaling in the JAK pathway (LNK or SH2B3); 3) translocations of tyrosine kinases leading to activated kinase signaling (BCR-ABL1, STRN3-JAK2, EBF1-PDGFRB, NUP214-ABL1, IGH@-EPOR, BCR-JAK2, PAX5-JAK2, ETV6-ABL1, RCSD1-ABL1, RANBP2-ABL1); and 4) all cases in the R8 cluster group which have been shown to be composed of cases containing a spectrum of mutations in various tyrosine kinases (as presented in attached Table 1a and Table 1b). While these categories are highly overlapping, the combination of the four affords the most inclusive model of tyrosine kinase related genomic mutations. We anticipate that this gene expression signature will be used as an initial screening test to prospectively identify Ph-like ALL patients who have a poor clinical outcome on standard regimens. Following this screening assay, secondary molecular assays (including PCR, sequencing, or FISH assays to identify specific mutations or translocations) or next generation sequencing methods under development for the clinical diagnostic setting may be used to identify the precise kinase mutation present in each case to best facilitate therapeutic targeting to TKIs or other interventions.

### SUMMARY OF THE INVENTION

In an embodiment, the invention provides a nucleic acid array for expression-based classification of B-precursor acute lymphoblastic leukemia (ALL) as being either responsive or non-responsive to tyrosine kinase inhibitor mono or co-therapy, the array comprising at least 5 probes, at least about 6-10 probes, about 10-50 probes up to about 100 or more probes, at least 6, 7, 8, 9, 10, 11, 12, 13, 14, 15, 16 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 probes immobilized on a solid support, each of the probes:

(a) having a length of between about 15-20 to about 500 or more nucleotides (up to several thousand nucleotide units, preferably about 20-25 to about 325-350 nucleotides, often 25-300 nucleotides); and
(b) being derived from sequences corresponding to, or complementary to, transcripts or partial transcripts of at least part of a 26 gene prognostic gene set of Table IV (see examples section) comprising at least IGJ, SPATS2L, MUCO, CRLF2 and CA6 (five genes) and optionally, at least one further gene (one or more) selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A of Table 4 hereof. In this aspect of the invention, a prognostic gene set corresponds to the first five genes set forth above and optionally one or more genes selected from the remaining genes (e.g., genes 6, 7, 8, 9, 10, 11, 12, 13, 14, 15, 16, 17, 18, 19, 20, 21, 22, 23, 24, 25 or 26, including the first 23, or all 26 genes) from the above gene set of Table 4 hereof.

As explained further hereinafter, the nucleic acid array(s) described above are used to determine an expression pattern profile for transcripts or partial transcripts of the gene set as described above. The transcripts or partial transcripts are derived from a sample taken from a subject suffering from B precursor acute lymphoblastic leukemia (ALL) and the expression pattern profile is compared to a reference expression pattern profile. A determination that the sample's expression levels of the gene sets as described above is equal to or exceeds its corresponding gene expression reference value indicates that the subject's B-precursor acute lymphoblastic leukemia (ALL) is responsive to tyrosine kinase inhibitor mono or co-therapy. A determination that the sample's expression level of the gene sets as described above is below its corresponding gene expression reference value indicates that the subject's B-precursor acute lymphoblastic leukemia (ALL) is likely to be non-responsive to tyrosine kinase inhibitor mono or co-therapy, and alternative therapy is proposed for that patient.

In another embodiment, the invention provides a nucleic acid array for expression-based classification of B-precursor acute lymphoblastic leukemia (ALL) as being either responsive or non-responsive to tyrosine kinase inhibitor mono or co-therapy, the array comprising at least 5 probes, at least about 10-50 probes up to about 100 or more probes, at least 11, 12, 13, 14, 15, 16 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 probes immobilized on a solid support, each of the probes:

(a) having a length of between about 15-20 to about 500 or more nucleotides (up to several thousand nucleotide units, preferably about 20-25 to about 325-350 nucleotides, often 25-300 nucleotides); and
(b) being derived from sequences corresponding to, or complementary to, transcripts or partial transcripts of each member of one or more of a first, second, third or fourth prognostic gene set, wherein:
(1) the first prognostic gene set consists essentially of IGJ, CRLF2, MUC4, SPATS2L, SLC2A5, PON2, CA6, NRXN3, DENND3, GPR110, BMPR1B and CD99;
(2) the second prognostic gene set consists essentially of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC;
(3) the third prognostic gene consists essentially of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and
(4) the fourth prognostic gene consists essentially of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157.

As explained further hereinafter, the nucleic acid array(s) described above are used to determine an expression pattern profile for transcripts or partial transcripts of each member of the one or more first, second, third or fourth prognostic gene sets. The transcripts or partial transcripts are derived from a sample taken from a subject suffering from B precursor acute lymphoblastic leukemia (ALL) and the expression pattern profile is compared to a reference expression pattern profile. A determination that the sample's expression levels of at least one member of the first, second, third or fourth gene sets is equal to or exceeds its corresponding gene expression reference value indicates that the subject's B-precursor acute lymphoblastic leukemia (ALL) is responsive to tyrosine kinase inhibitor mono or co-therapy. A determination that the sample's expression level of the gene sets as described above is below its corresponding gene expression reference value indicates that the subject's B-precursor acute lymphoblastic leukemia (ALL) is likely to be non-responsive to tyrosine kinase inhibitor mono or co-therapy, and alternative therapy is proposed for that patient.

In certain embodiments, the probe sequences hybridize under stringent or non-stringent conditions to mRNA corresponding to each member of one or more of the first, second, third or fourth prognostic gene sets. In other embodiments, the probe sequences hybridize under stringent or non-stringent conditions to cDNA corresponding to each member of one or more of the first, second, third or fourth prognostic gene sets.

In another embodiment, the invention provides a method of classifying a subject's B precursor acute lymphoblastic leukemia (ALL) as being either responsive or non-responsive to tyrosine kinase inhibitor mono or co-therapy, the method comprising:

(a) determining the expression level in a sample obtained from the subject of transcripts or partial transcripts of at least five genes (IGJ, SPATS2L, MUC4, CRLF2 and CA6) and optionally, at least one and up to 21 further genes selected from the group consisting of NRXN3; BMPR1B; GPR110; SEMA6A; PON2; CHN2; S100Z; SLC2A5; TP5i3INP1; IFITM1; GBP5; TMEM154; CD99; MDFIC; LDB3; TTYH2; DENND3; SLC37A3; ENAM; LOC645744 and WNT9A as described above, thereby deriving an expression pattern profile; and
(b) comparing the expression pattern profile to a reference expression pattern profile; wherein a determination that the sample's expression levels of the prognostic gene set as described above is equal to or exceeds its corresponding gene expression reference value indicates that the subject's B-precursor acute lymphoblastic leukemia (ALL) is responsive to tyrosine kinase inhibitor mono or co-therapy.

In another alternative embodiment, the invention provides a method of classifying a subject's B precursor acute lymphoblastic leukemia (ALL) as being either responsive or non-responsive to tyrosine kinase inhibitor mono or co-therapy, the method comprising:

(a) determining the expression level in a sample obtained from the subject of transcripts or partial transcripts of each member of one or more of the first, second, third or fourth prognostic gene sets described above, thereby deriving an expression pattern profile; and
(b) comparing the expression pattern profile to a reference expression pattern profile;
wherein a determination that the sample's expression levels of at least one member of the first, second, third or fourth gene sets is equal to or exceeds its corresponding gene expression reference value indicates that the subject's B-precursor acute lymphoblastic leukemia (ALL) is responsive to tyrosine kinase inhibitor mono or co-therapy.

In certain embodiments, derivation of the expression pattern profile and comparison of the expression pattern profile to the reference expression pattern profile involves application of an algorithm to expression level values of the transcripts or partial transcripts to the appropriate gene set. Typically, a comparison of the expression pattern profile to a reference expression pattern profile which shows an increased level of expression of the transcripts or partial transcripts of the prognostic gene sets (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above) indicates that the subject's B-precursor acute lymphoblastic leukemia (ALL) is responsive to tyrosine kinase inhibitor mono or co-therapy.

In certain embodiments, the step of determining the expression level of the transcripts or partial transcripts of the genes to be measured (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above) involves preparation from the sample of mRNA corresponding to the genes to be measured in the prognostic gene sets. In other embodiments, the mRNA is amplified by quantitative PCR to produce cDNA. In still other embodiments, the mRNA is amplified by reverse transcription PCR (RT-PCR) to produce cDNA. The step of determining the expression level of the transcripts or partial transcripts of each gene to be measured can also involve preparation from the sample of polypeptides encoded by each member of the prognostic gene set. Polypeptide expression levels can be determined by antibody detection or other techniques that are well-known to those of ordinary skill in the art.

In another embodiment, the invention provides a system for expression-based classification of B-precursor acute lymphoblastic leukemia (ALL) as being either responsive or non-responsive to tyrosine kinase inhibitor mono or co-therapy, the system comprising polynucleotide sequences corresponding to, or complementary to, transcripts or partial transcripts of each member of the gene set(s) to be measured as described above (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above). The polynucleotide sequences used in these systems can also hybridize under stringent or non-stringent conditions to mRNA transcripts or mRNA partial transcripts of each member of the gene set(s) to be measured. Or the polynucleotide sequences can hybridize under stringent or non-stringent conditions to cDNA transcripts or cDNA partial transcripts of each member of the gene set(s) to be measured.

In still another embodiment, the invention provides a computer-readable medium comprising one or more digitally-encoded expression pattern profiles representative of the level of expression of transcripts or partial transcripts of each member of the prognostic gene set(s) to be measured as described above (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above). Each of the one or more expression pattern profiles is associated with a value that is correlated with a reference expression pattern profile to yield a predictor of whether a subject's B-precursor acute lymphoblastic leukemia (ALL) is responsive to tyrosine kinase inhibitor mono or co-therapy.

In still another embodiment, the invention provides a method of determining whether a subject's B-precursor acute lymphoblastic leukemia (ALL) is responsive to tyrosine kinase inhibitor mono or co-therapy, the method comprising:

(a) assaying a sample obtained from the subject to determine the expression level of transcripts or partial transcripts of at least part of a 26 gene prognostic gene set comprising at least the genes IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A, thereby deriving an expression pattern profile; and
(b) comparing the expression pattern profile to a reference expression pattern profile. wherein a comparison of the expression pattern profile to a reference expression pattern profile which shows an increased level of expression of the transcripts or partial transcripts of the genes of the prognostic gene sets to be measured indicates that the subject's B-precursor acute lymphoblastic leukemia (ALL) is responsive to tyrosine kinase inhibitor mono or co-therapy. In additional embodiments, depending upon the patient's prognosis, tyrosine kinase monotherapy or co-therapy is administered to the patient to enhance the therapeutic outcome. In instances where the method evidences that the patient will not have a favorable prognosis with tyrosine kinase monotherapy or co-therapy, a more aggressive chemotherapeutic regimen may be administered (monotherapy or co-therapy as described above, but with more aggressive therapeutic intervention, e.g. substantially higher doses of tyrosine kinase inhibitor monotherapy or co-therapy or an alternative therapy, including experimental therapies).

In still another embodiment, the invention provides a method of determining whether a subject's B-precursor acute lymphoblastic leukemia (ALL) is responsive to tyrosine kinase inhibitor mono or co-therapy, the method comprising:

(a) assaying a sample obtained from the subject to determine the expression level of transcripts or partial transcripts of each member of one or more of the first, second, third or fourth prognostic gene sets described above, thereby deriving an expression pattern profile; and
(b) comparing the expression pattern profile to a reference expression pattern profile.
wherein a comparison of the expression pattern profile to a reference expression pattern profile which shows an increased level of expression of the transcripts or partial transcripts of each member of one or more of the first, second, third or fourth prognostic gene sets indicates that the subject's B-precursor acute lymphoblastic leukemia (ALL) is responsive to tyrosine kinase inhibitor mono or co-therapy. In additional embodiments, depending upon the patient's prognosis, tyrosine kinase monotherapy or co-therapy is administered to the patient to enhance the therapeutic outcome. In instances where the method evidences that the patient will not have a favorable prognosis with tyrosine kinase monotherapy or co-therapy, a more aggressive chemotherapeutic regimen may be administered (monotherapy or co-therapy as described above, but with more aggressive therapeutic intervention, e.g. substantially higher doses of tyrosine kinase inhibitor monotherapy or co-therapy or an alternative therapy, including experimental therapies).

In certain embodiments, assaying of the sample comprises gene expression by an array. Assaying of the sample can also comprise preparing mRNA from the sample; the mRNA can be amplified by quantitative PCR to produce cDNA. mRNA can also be amplified by reverse transcription PCR (RT-PCR) to produce cDNA.

One or more of the steps of the methods described herein can be performed in silica.

Representative, non-limiting samples include samples of bone marrow or peripheral blood.

In still another embodiment, the invention provides a kit for characterizing the expression level of transcripts or partial transcripts of each member of prognostic gene set(s) described above to be measured (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above), the kit comprising:

(a) each member of the prognostic gene set to be measured or a complement thereto; and/or
(b) mRNA forms of each member of a prognostic gene set to be measured or a complement thereto; and/or
(c) polypeptides encoded by each member of the prognostic gene set to be measured or a complement thereto; and optionally
(d) instructions for correlating the expression level of (i) each member of the prognostic gene set to be measured or a complement thereto, and/or
(ii) mRNA forms of each member of the prognostic gene set to be measured or a complement thereto, and/or (iii) polypeptides encoded by each member of the prognostic gene set to be measured or a complement thereto with the effectiveness of tyrosine kinase inhibitor mono or co-therapy in treating B-precursor acute lymphoblastic leukemia (ALL).

In still another embodiment, the invention provides a device for determining whether a B-precursor acute lymphoblastic leukemia (ALL) is responsive to tyrosine kinase inhibitor mono or co-therapy, the device comprising:

(a) means for measuring the expression level of transcripts or partial transcripts of each member of the prognostic gene set to be measured (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above);
(b) means for correlating the expression level with a classification of B-precursor acute lymphoblastic leukemia (ALL) status; and
(c) means for outputting the B-precursor acute lymphoblastic leukemia (ALL) status; wherein the device optionally utilizes an algorithm to characterize the expression level.

Preferably, the reference expression pattern profile is determined by application of an algorithm to control sample expression level values of transcripts or partial transcripts of each member of the prognostic gene set to be measured (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above). Details regarding non-limiting useful algorithms are provided hereinafter. As described in more detail below, a useful algorithm can be generated by kinase prediction modeling of a B-precursor acute lymphoblastic leukemia (ALL) patient training set using the Prediction Analysis of Microarray (PAM) method and the following three separate optimization criteria: average error, overall error and AUC.

These and other aspects of the invention are described further in the Detailed Description of the Invention.

### BRIEF DESCRIPTION OF THE FIGURES

FIG. 1. Determination of Optimal Number of Microarray Probe Sets by Three Methods. FIG. 1 illustrates the determination of the optimal number of microarray probe sets by three methods that are explained in further detail in the examples.

FIG. 2. Predictions of 42 Probe Set Model in the Test Set. FIG. 2 illustrates predictions of a 42 probe set model in the test set explained in further detail in the examples.

FIG. 3. Determination of Optimal Number of LDA Genes by Three Methods. FIG. 3 illustrates the determination of the optimal number of LDA genes by three methods that are explained in further detail in the examples.

FIGS. 4A and B. LDA Model Performance in Test Set. FIGS. 4A and B illustrate a LDA model performance in a test set, as explained in the examples.

FIG. 5. Survival Plots of Training Set Using Array Models. FIG. 5 illustrates survival plots of training sets using array models, as described in the examples.

FIG. 6. Survival Plots of Training Sets Using LDA Models. FIG. 6 illustrates survival plots of training sets using LDA models, as described in the examples.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The term “at least one further” describes one or more of the enumerated species which is set forth after that term in a phrase. Thus, for example, a preferred prognostic gene set for use in the present invention, in various aspects, is derived from the 26 gene prognostic gene set of Table IV (see examples section) and generally comprising at least IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A, as those genes are set forth in Table 4 hereof. In this aspect, the term “at least one further gene” includes one or more genes selected from the remaining genes of Table 4 (e.g., any one or more of genes 6, 7, 8, 9, 10, 11, 12, 13, 14, 15, 16, 17, 18, 19, 20, 21, 22, 23, 24, 25 or 26 from the gene set of Table 4).

Furthermore, the following terms shall have the definitions set out below.

The term “high risk B precursor acute lymphocytic leukemia” or “high risk B-ALL” refers to a disease state of a patient with acute lymphoblastic leukemia who meets certain high risk disease criteria. These include: confirmation of B-precursor ALL in the patient by central reference laboratories (See Borowitz, et al., Rec Results Cancer Res 1993; 131: 257-267); and exhibiting a leukemic cell DNA index of ≦1.16 (DNA content in leukemic cells: DNA content of normal \tilde{G}₀G₁ cells) (DI) by central reference laboratory (See, Trueworthy, et al., J Clin Oncol 1992; 10: 606-613; and Pullen, et al., “Immunologic phenotypes and correlation with treatment results”. In Murphy S B, Gilbert J R (eds). Leukemia Research: Advances in Cell Biology and Treatment. Elsevier: Amsterdam, 1994, pp 221-239) and at least one of the following: (1) WBC≧10 000-99 000/μl, aged 1-2.99 years or ages 6-21 years; (2) WBC≧100 000/μl, aged 1-21 years; (3) all patients with CNS or overt testicular disease at diagnosis; or (4) leukemic cell chromosome translocations t(1;19) or t(9;22) confirmed by central reference laboratory. (See, Crist, et al, Blood 1990; 76: 117-122; and Fletcher, et al., Blood 1991; 77: 435-439).

The term “patient” shall mean within context an animal, preferably a mammal, more preferably a human patient, more preferably a human child who is undergoing or will undergo therapy or treatment for leukemia, especially high risk B-precursor acute lymphoblastic leukemia.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (eg., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis. A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell. A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell.

Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

This stability is demonstrated by the ability of the eukaryotic cell to establish cell lines or clones comprised of a population of daughter cells containing the transforming DNA.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, “epitope” refers to an antigenic determinant of a polypeptide. An epitope could comprise 3 amino acids in a spatial conformation which is unique to the epitope. Generally an epitope consists of at least 5 such amino acids, and more usually, consists of at least 8-10 such amino acids. Methods of determining the spatial conformation of amino acids are known in the art, and include, for example, x-ray crystallography and 2-dimensional nuclear magnetic resonance.

As used herein, a “mimotope” is a peptide that mimics an authentic antigenic epitope.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

Sequence data for each member of the first, second, third and fourth prognostic gene set may be found at a number of sources available to those of ordinary skill in the art, including but not limited to the NIH GENBANK® database and the NCBI Entrez Gene database. These are all well-known in the art.

As used herein, “antibody” includes, but is not limited to, monoclonal antibodies. The following disclosure from U.S. Patent Application Document No. 20100284921, the entire contents of which are hereby incorporated by reference, exemplifies techniques that are useful in making antibodies employed in formulations of the instant invention.

As described in U.S. Patent Application Document No. 20100284921, “antibodies . . . may be polyclonal or monoclonal. Monoclonal antibodies are preferred. The antibody is preferably a chimeric antibody. For human use, the antibody is preferably a humanized chimeric antibody.

[A]n anti-target-structure antibody . . . may be monovalent, divalent or polyvalent in order to achieve target structure binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes [use of] functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application Nos. WO 1993/21319 and WO 1989/09622. Functional equivalents include polypeptides with amino acid sequences substantially the same as the amino acid sequence of the variable or hypervariable regions of the antibodies raised against target integrins according to the practice of the present invention.

Functional equivalents of the anti-target-structure antibodies further include fragments of antibodies that have the same, or substantially the same, binding characteristics to those of the whole antibody. Such fragments may contain one or both Fab fragments or the F(ab′).sub.2 fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the target structure binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies (e.g. for preparing an antibody or an antigen binding fragment thereof having specific binding affinity for either caspase-1 or an autophagy-related immunomodulatory cytokine) are either described in the experiments herein or are otherwise known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)2 fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with a target structure or a fragment thereof. Antibodies produced in the inoculated animal that specifically bind the target structure are then isolated from fluid obtained from the animal. Anti-target-structure antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.).

When the antibody used in the methods used in the practice of the invention is a monoclonal antibody, the antibody is generated using any well known monoclonal antibody preparation procedures such as those described, for example, in Harlow et al. (supra) and in Tuszynski et al. (Blood 1988, 72:109-115). Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (supra).

Chimeric animal-human monoclonal antibodies may be prepared by conventional recombinant DNA and gene transfection techniques well known in the art. The variable region genes of a mouse antibody-producing myeloma cell line of known antigen-binding specificity are joined with human immunoglobulin constant region genes. When such gene constructs are transfected into mouse myeloma cells, the antibodies produced are largely human but contain antigen-binding specificities generated in mice. As demonstrated by Morrison et al., 1984, Proc. Natl. Acad. Sci. USA 81:6851-6855, both chimeric heavy chain V region exon (VH)-human heavy chain C region genes and chimeric mouse light chain V region exon (VK)-human K light chain gene constructs may be expressed when transfected into mouse myeloma cell lines. When both chimeric heavy and light chain genes are transfected into the same myeloma cell, an intact H2L2 chimeric antibody is produced. The methodology for producing such chimeric antibodies by combining genomic clones of V and C region genes is described in the above-mentioned paper of Morrison et al., and by Boulianne et al. (Nature 1984, 312:642-646). Also see Tan et al. (J. Immunol. 1985, 135:3564-3567) for a description of high level expression from a human heavy chain promotor of a human-mouse chimeric K chain after transfection of mouse myeloma cells. As an alternative to combining genomic DNA, cDNA clones of the relevant V and C regions may be combined for production of chimeric antibodies, as described by Whitte et al. (Protein Eng. 1987, 1:499-505) and Liu et al. (Proc. Natl. Acad. Sci. USA 1987, 84:3439-3443). For examples of the preparation of chimeric antibodies, see the following U.S. Pat. Nos. 5,292,867; 5,091,313; 5,204,244; 5,202,238; and 5,169,939. The entire disclosures of these patents, and the publications mentioned in the preceding paragraph, are incorporated herein by reference. Any of these recombinant techniques are available for production of rodent/human chimeric monoclonal antibodies against target structures.

To further reduce the immunogenicity of murine antibodies, “humanized” antibodies have been constructed in which only the minimum necessary parts of the mouse antibody, the complementarity-determining regions (CDRs), are combined with human V region frameworks and human C regions (Jones et al., 1986, Nature 321:522-525; Verhoeyen et al., 1988, Science 239:1534-1536; Hale et al., 1988, Lancet 2:1394-1399; Queen et al., 1989, Proc. Natl. Acad. Sci. USA 86:10029-10033). The entire disclosures of the aforementioned papers are incorporated herein by reference. This technique results in the reduction of the xenogeneic elements in the humanized antibody to a minimum. Rodent antigen binding sites are built directly into human antibodies by transplanting only the antigen binding site, rather than the entire variable domain, from a rodent antibody. This technique is available for production of chimeric rodent/human anti-target structure antibodies of reduced human immunogenicity.”

A “primer” or “probe” of the present invention is typically at least about 15-20 nucleotides in length. In one embodiment of the invention, a primer or a probe is at least about 20-25 to about 500, about 20-25 to about 350 nucleotides in length, about 25-300 nucleotides, about 25 to about 100 nucleotides, about 25 to about 50 in length. In a preferred embodiment, a primer or a probe is at least about 25-30 nucleotides in length. While the maximal length of a probe can be as long as the target sequence to be detected, depending on the type of assay in which it is employed, it is typically less than about 500 nucleotide units in length, preferably less than about 350 nucleotide units in length, less than about 325 nucleotide units in length, less than about 300 nucleotide units in length. In the case of a primer, it is typically less than about 30-35 nucleotides in length. In a specific preferred embodiment of the invention, a primer or a probe is within the length of about 25 and about 50 nucleotides. However, in other embodiments, such as nucleic acid arrays and other embodiments in which probes are affixed to a substrate, the probes can be longer, such as on the order of 100-500 or more (up to several thousand or more) nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Pre-hybridization with a solution containing 5 times standard saline phosphate EDTA (SSPE), 0.5% NaDodSO.sub.4 (SDS) at 55° C., and incubating probe with target nucleic acid molecules in the same solution at the same temperature, followed by washing with a solution containing 2 times SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment which is particularly suitable for use in a oligonucleotide ligation assay (U.S. Pat. No. 4,988,617), the 3′ most nucleotide of the probe aligns with the SNP position in the target sequence.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

The term “stringent hybridization conditions” are known to those skilled in the art and can be found in Current Protocols in Molecular Biology, John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2.×SSC, 0.1% SDS at 50° C., preferably at 55° C., and purely by way of example, a comparison of the expression pattern profile to a reference expression pattern profile which shows differences in the level of expression of the transcripts or partial transcripts of each member of one or more of the first, second, third or fourth prognostic gene sets can reflect expression level differences of about ±50% to about ±0.5%, or about ±45% to about ±1%, or about ±40% to about ±1.5%, or about ±35% to about ±2.0%, or about +30% to about +2.5%, or about f 25% to about ±3.0%, or about ±20% to about +3.5%, or about ±15% to about ±4.0%, or about ±10% to about +5.0%, or about ±9% to about ±1.0%, or about +8% to about ±2%, or about ±7% to about +3%, or about ±6% to about ±5%, or about ±5%, or about +4.5%, or about +4.0%, or about ±3.5%, or about +3.0%, or about ±2.5%, or about ±2.0%, or about ±1.5%, or about ±1.0%.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002; 8:85-101; Sosnowski et al., “Active microelectronic array system for DNA hybridization, genotyping and pharmacogenomic applications”, Psychiatr Genet. 2002 December; 12(4):181-92; Heller, “DNA microarray technology: devices, systems, and applications”, Annu Rev Biomed Eng. 2002; 4: 129-53. Epub 2002 Mar. 22; Kolchinsky et al., “Analysis of SNPs and other genomic variations using gel-based chips”, Hum Mutat. 2002 April; 19(4):343-60; and McGall et al., “High-density genechip oligonucleotide probe arrays”, Adv Biochem Eng Biotechnol. 2002; 77:21-42.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 20-25 to about 500 or more (up to several thousand) nucleotides in length, more preferably about 25 to about 350 nucleotides in length, and often about 25-100 nucleotides or 25 to about 50 nucleotides in length. For certain types of microarrays or other detection kits/systems, it may be preferable to use oligonucleotides that are only about 20-30, preferably about 25 nucleotides in length.

In other types of arrays, such as arrays used in conjunction with chemiluminescent detection technology, preferred probe lengths can be, for example, about 20-25 to several thousand nucleotides in length, preferably about 25 to about 500 nucleotides in length, often about 100 to 500 nucleotides in length, and often about 50 to about 350 nucleotides in length. The microarray or detection kit can contain polynucleotides that cover the known 5′ or 3′ sequence of a gene/transcript or target, sequential polynucleotides that cover the full-length sequence of a gene/transcript; or unique polynucleotides selected from particular areas along the length of a target gene/transcript sequence. Polynucleotides used in the microarray or detection kit can be specific to a gene/transcript or target of interest.

Hybridization assays based on polynucleotide arrays rely on the differences in hybridization stability of the probes to perfectly matched and mismatched target sequence variants. It is generally preferable that stringency conditions used in hybridization assays are high enough such that nucleic acid molecules that differ from one another at as little as a single gene/transcript or target position can be differentiated. Representative high stringency conditions are described herein and well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 20-25 to about 500 or more nucleotides in length. In further embodiments, a nucleic acid array can comprise any number of probes, in which at least one probe is capable of detecting one or more sequences described herein, or a fragment of such sequences comprising at least about 20-25 consecutive nucleotides, preferably about 25 to about 350, often about 25 to about 100 or more consecutive nucleotides (or any other number in-between).

In another embodiment, a “probe set” can be designed (pursuant to the ID as listed in the tables set forth herein) on arrays to span approximately 300 or so bases of the gene, typically in the 3′ untranslated regions, although they may also cover some exons. The design of 99% of these probe sets involves 12 “perfect match” oligos, each of which is about 25 bases long. If these don't overlap, this would cover 300 bases of the target gene. For the most part, it is certainly possible that a single oligo of this probe set would be capable of identifying the expression of the gene. Commercialization efforts often center on the use of 25 in order to boost the signal and try to work around cross-hybridization issues and polymorphisms. This approach increases the signal by adding more probes.

In still another embodiment, LDA gene assays involve two primers and a non-overlapping probe between them. Primers in this application are usually in the range of about 20-25 bases long and TaqMan probes are typically slightly larger, around 30 bases (the Taq Man system requires that the probes anneal first, which is usually accomplished by making them longer). By providing amplification that is 100% efficiency, this will double the amount of target at every cycle. When the amplication cycle begins at each cycle the material is melted and then the primer/probe starts annealing. If the probe anneal first, followed by the upstream primer, then the polymerase/nuclease features of the PCR enzymes will chew the labels off of the probes as the amplicon is being made. Since the probe has both a fluor and a quencher, when they are in close proximity (i.e. attached to the probe) there is no fluorescence. As soon as the enzyme chews it off, the fluorescent moiety emits light. At the end of each cycle the fluorescence is measured and the increase in fluorescence is a directed measure of the amount of product made. This process may be repeated, e.g. for 40 cycles. The specificity of this method is conferred by the fact that two separate primers are necessary to make the product, and a non-overlapping probe detects it. The method is quite efficient and highly quantitative and specific. It is a single probe system, but quantitatively amplifies the product to determine the initial target amount.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be/used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain at least about 5 polynucleotides, at least about 6-10 polynucleotides, about 10-50 polynucleotides, up to about 100 or more polynucleotides, about 12 to about 42 or more polynucleotides, or any other number which lends itself to the efficient use of commercially available instrumentation.

As indicated above, reference expression pattern profiles are preferably determined by application of an algorithm to control sample expression level values of transcripts or partial transcripts of each member of the prognostic gene set(s) (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above). In non-limiting examples, such algorithms can be derived as shown in the examples herein and may be optimization algorithms such as a mean variance algorithm, and/or may be heuristic, and or may be a repeatability based meta-analysis classification algorithm, and/or may be a classifier algorithm.

In certain embodiments, illustrative algorithms include but are not limited to methods that reduce the number of variables such as principal component analysis algorithms, partial least squares methods, and independent component analysis algorithms. Illustrative algorithms further include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof. In some embodiments of the present invention a support vector machine (SVM) algorithm, a random forest algorithm, or a combination thereof is used for classification of microarray data. In some embodiments, identified markers that distinguish samples or subtypes are selected based on statistical significance. In some cases, the statistical significance selection is performed after applying a Benjamini Hochberg correction for false discovery rate (FDR).

Those of ordinary skill in the art know how to apply the aforementioned and other algorithmic techniques to the members of the prognostic gene sets (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above) to derive useful algorithms.

In some cases, the classifier algorithm may be supplemented with a meta-analysis approach such as that described by Fishel and Kaufman et al. 2007 Bioinformatics 23(13): 1599-606. Also, the classifier algorithm may be supplemented with a meta-analysis approach such as a repeatability analysis. In some cases, the repeatability analysis selects markers that appear in at least one predictive expression product marker set.

The practice of the present invention may also employ conventional biology methods, software and systems. For example, means for measuring the expression level of transcripts or partial transcripts of each member of the prognostic gene set(s) (for example, at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A or each member of one or more of a first, second, third or fourth prognostic gene set as described above); means for correlating the expression level with a classification of B-precursor acute lymphoblastic leukemia (ALL) status; and means for outputting the B-precursor acute lymphoblastic leukemia (ALL) status may employ conventional biology methods, software and systems as described herein or as otherwise known to those of ordinary skill in the art.

Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing information over networks such as the Internet. For example, the components of the system may be interconnected via any suitable means including over a network, e.g. the ELISA plate reader to the processor or computing device. The processor may take the form of a portable processing device that may be carried by an individual user e.g. lap top, and data can be transmitted to or received from any device, such as for example, server, laptop, desktop, PDA, cell phone capable of receiving data, BLACKBERRY®, and the like. In some embodiments of the invention, the system and the processor may be integrated into a single unit. In another example, a wireless device can be used to receive information and forward it to another processor over a telecommunications network, for example, a text or multi-media message.

The functions of the processor need not be carried out on a single processing device. They may, instead be distributed among a plurality of processors, which may be interconnected over a network. Further, the information can be encoded using encryption methods, e.g. SSL, prior to transmitting over a network or remote user. The information required for decoding the captured encoded images taken from test objects may be stored in databases that are accessible to various users over the same or a different network.

In some embodiments, the data is saved to a data storage device and can be accessed through a web site. Authorized users can log onto the web site, upload scanned images, and immediately receive results on their browser. Results can also be stored in a database for future reviews.

In some embodiments, a web-based service may be implemented using standards for interface and data representation, such as SOAP and XML, to enable third parties to connect their information services and software to the data. This approach would enable seamless data request/response flow among diverse platforms and software applications.

“Tyrosine kinase inhibitors” include, but are not limited to imatinib, axitinib, bosutinib, cediranib, dasatinib, erlotinib, gefitinib, lapatinib, lestaurtinib, nilotinib, semaxanib, sunitinib, toceranib, vandetanib, vatalanib, sorafenib (Nexavar®), lapatinib, motesanib, vandetanib (Zactima®), MP-412, lestaurtinib, XL647, XL999, tandutinib, PKC412, AEE788, OSI-930, OSI-817, sunitinib maleate (Sutent®)) and N-(4-(4-aminothieno[2,3-d]pyrimidin-5-yl)phenyl)-N-(2-fluoro-5-(trifluor-omethyl)phenyl)urea, the preparation of which is described in United States Patent Application Document No. 2007/0155758.

The term “tyrosine kinase inhibitors” is intended to encompass the hydrates, solvates (such as alcoholates), polymorphs, N-oxides, and pharmaceutically acceptable acid or base addition salts of tyrosine kinase inhibiting compounds. The term “tyrosine kinase inhibitor mono therapy” is used to describe a treatment regimen wherein one or more tyrosine kinase inhibitors (in the absence of other chemotherapeutic agents, etc.) is administered to a patient to treat cancer who has shown, by application of the present invention, to have a likelihood of a favorable prognosis on such therapy. The term “tyrosine kinase inhibitor cotherapy” is used to describe therapy which comprises administering at least one tyrosine kinase inhibitor as otherwise described herein and traditional therapy, described below.

The term “traditional therapy” is directed to therapy (protocol) which is typically used to treat leukemia, especially B-precursor ALL (including pediatric B-ALL) and can include Memorial Sloan-Kettering New York II therapy (NY II), UKALLR2, AL 841, AL851, ALHR88, MCP841 (India), as well as modified BFM (Berlin-Frankfurt-Munster) therapy, BMF-95 or other therapy, including ALinC 17 therapy as is well-known in the art. In the present invention the term “more aggressive therapy” or “alternative therapy” usually means a more aggressive version of tyrosine kinase monotherapy, tyrosine kinase cotherapy or more conventional therapy typically used to treat leukemia, for example B-ALL, including pediatric B-precursor ALL, using for example, conventional or traditional chemotherapeutic agents at higher dosages and/or for longer periods of time in order to increase the likelihood of a favorable therapeutic outcome. It may also refer, in context, to experimental therapies for treating leukemia, rather than simply more aggressive versions of conventional (traditional) therapy.

The term “effective” is used herein, unless otherwise indicated, to describe an amount of a compound or composition which, in context, is used to produce or affect an intended result, whether that result relates to treating a subject who suffers from cancer and symptoms and conditions associated with cancer. This term subsumes all other effective amount or effective concentration terms which are otherwise described in the present application.

The term “inhibitory effective concentration” or “inhibitory effective amount” describes concentrations or amounts of compounds that, when administered according to the present invention, substantially or significantly inhibit aspects or symptoms of cancer or conditions associated with cancer.

The term “preventing effective amount” describes concentrations or amounts of compounds which, when administered according to the present invention, are prophylactically effective in preventing or reducing the likelihood of the onset of cancer or a condition associated with cancer or in ameliorating the symptoms of such disorders or symptoms. The terms inhibitory effective amount or preventive effective amount also generally fall under the rubric “effective amount”.

In certain embodiments, a B-precursor acute lymphoblastic leukemia (ALL) is predicted to be either responsive or non-responsive to tyrosine kinase inhibitor mono or co-therapy based on a determination of whether it is likely to result in one or more of the clinical outcomes outlined in the following excerpts from the National Cancer Institute Childhood Acute Lymphoblastic Leukemia Treatment (PDQ®) (http://www.cancer.gov/cancertopics/pdq/treatment/childALL/HealthProfessional/Page2#Section_526). (These clinical assessments and prognosis indicia are purely exemplary and are not limiting. Other clinical analyses may be employed in the determination of whether a B-precursor acute lymphoblastic leukemia (ALL) will respond to tyrosine kinase inhibitor mono or co-therapy.)

The rapidity with which leukemia cells are eliminated following onset of treatment and the level of residual disease at the end of induction are associated with long-term outcome. Because treatment response is influenced by the drug sensitivity of leukemic cells and host pharmacodynamics and pharmacogenomics, early response has strong prognostic significance. Various ways of evaluating the leukemia cell response to treatment have been utilized, including the following:
1. MRD determination.
2. Day 7 and day 14 bone marrow responses.
3. Peripheral blood response to steroid prophase.
4. Peripheral blood response to multiagent induction therapy.
5. Induction failure.

### MRD Determination.

Morphologic assessment of residual leukemia in blood or bone marrow is often difficult and is relatively insensitive. Traditionally, a cutoff of 5% blasts in the bone marrow (detected by light microscopy) has been used to determine remission status. This corresponds to a level of 1 in 20 malignant cells. If one wishes to detect lower levels of leukemic cells in either blood or marrow, specialized techniques such as PCR assays, which determine unique IgIT-cell receptor gene rearrangements, fusion transcripts produced by chromosome translocations, or flow cytometric assays, which detect leukemia-specific immunophenotypes, are required. With these techniques, detection of as few as 1 leukemia cell in 100,000 normal cells is possible, and MRD at the level of 1 in 10,000 cells can be detected routinely.

Multiple studies have demonstrated that end-induction MRD is an important, independent predictor of outcome in children and adolescents with B-lineage ALL. MRD response discriminates outcome in subsets of patients defined by age, leukocyte count, and cytogenetic abnormalities. Patients with higher levels of end-induction MRD have a poorer prognosis than those with lower or undetectable levels. End-induction MRD is used by almost all groups as a factor determining the intensity of postinduction treatment, with patients found to have higher levels allocated to more intensive therapies. MRD levels at earlier (e.g., day 8 and day 15 of induction) and later time points (e.g., week 12 of therapy) also predict outcome.

MRD measurements, in conjunction with other presenting features, have also been used to identify subsets of patients with an extremely low risk of relapse. The COG reported a very favorable prognosis (5-year EFS of 97%±1%) for patients with B-precursor phenotype, NCI standard risk age/leukocyte count, CNS1 status, and favorable cytogenetic abnormalities (either high hyperdiploidy with favorable trisomies or the ETV6-RUNX1 fusion) who had less than 0.01% MRD levels at both day 8 (from peripheral blood) and end-induction (from bone marrow).

There are fewer studies documenting the prognostic significance of MRD in T-cell ALL. In the AIEOP-BFM ALL 2000 trial, MRD status at day 78 (week 12) was the most important predictor for relapse in patients with T-cell ALL. Patients with detectable MRD at end-induction who had negative MRD by day 78 did just as well as patients who achieved MRD-negativity at the earlier end-induction time point. Thus, unlike in B-cell precursor ALL, end-induction MRD levels were irrelevant in those patients whose MRD was negative at day 78. A high MRD level at day 78 was associated with a significantly higher risk of relapse.

There are few studies of MRD in the CSF. In one study, MRD was documented in about one-half of children at diagnosis. In this study, CSF MRD was not found to be prognostic when intensive chemotherapy was given.

Although MRD is the most important prognostic factor in determining outcome, there are no data to conclusively show that modifying therapy based on MRD determination significantly improves outcome in newly diagnosed ALL.

### Day 7 and Day 14 Bone Marrow Responses.

Patients who have a rapid reduction in leukemia cells to less than 5% in their bone marrow within 7 or 14 days following initiation of multiagent chemotherapy have a more favorable prognosis than do patients who have slower clearance of leukemia cells from the bone marrow.

### Peripheral Blood Response to Steroid Prophase.

Patients with a reduction in peripheral blast count to less than 1,000/μl after a 7-day induction prophase with prednisone and one dose of intrathecal methotrexate (a good prednisone response) have a more favorable prognosis than do patients whose peripheral blast counts remain above 1,000/μL (a poor prednisone response). Poor prednisone response is observed in fewer than 10% of patients. Treatment stratification for protocols of the Berlin Frankfurt-Münster (BFM) clinical trials group is partially based on early response to the 7-day prednisone prophase (administered immediately prior to the initiation of multiagent remission induction).

Patients with no circulating blasts on day 7 have a better outcome than those patients whose circulating blast level is between 1 and 999/μL.

### Peripheral Blood Response to Multiagent Induction Therapy.

Patients with persistent circulating leukemia cells at 7 to 10 days after the initiation of multiagent chemotherapy are at increased risk of relapse compared with patients who have clearance of peripheral blasts within 1 week of therapy initiation.[151] Rate of clearance of peripheral blasts has been found to be of prognostic significance in both T-cell and B-lineage ALL.

### Induction Failure.

The vast majority of children with ALL achieve complete morphologic remission by the end of the first month of treatment. The presence of greater than 5% lymphoblasts at the end of the induction phase is observed in up to 5% of children with ALL.[152] Patients at highest risk of induction failure have one or more of the following features:
T-cell phenotype (especially without a mediastinal mass).
B-precursor ALL with very high presenting leukocyte counts.
11q23 rearrangement.
Older age.
Philadelphia chromosome.

In a large retrospective study, the OS of patients with induction failure was only 32%. However, there was significant clinical and biological heterogeneity. A relatively favorable outcome was observed in patients with B-precursor ALL between the ages of 1 and 5 years without adverse cytogenetics (MLLtranslocation or BCR-ABL). This group had a 10-year survival exceeding 50%, and SCT in first remission was not associated with a survival advantage compared with chemotherapy alone for this subset. Patients with the poorest outcomes (<20% 10-year survival) included those who were aged 14 to 18 years, or who had the Philadelphia chromosome or MLL rearrangement. B-cell ALL patients younger than 6 years and T-cell ALL patients (regardless of age) appeared to have better outcomes if treated with allogeneic SCT after achieving complete remission than those who received further treatment with chemotherapy alone.

The term “patient” or “subject” is used throughout the specification within context to describe an animal, generally a mammal and preferably a human, to whom treatment, including prophylactic treatment, according to the present invention is provided. For treatment of symptoms which are specific for a specific animal such as a human patient, the term patient refers to that specific animal.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the term “neoplasia” is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be a tyrosine kinase inhibitor that is different from a tyrosine kinase inhibitor which has been previously administered to a subject. In many instances, the co-administration of another anti-cancer compound results in a synergistic anti-cancer effect.

Exemplary anti-cancer compounds for co-administration according to the present invention include anti-metabolites agents which are broadly characterized as antimetabolites, inhibitors of topoisomerase I and II, alkylating agents and microtubule inhibitors (e.g., taxol), as well as, EGF kinase inhibitors (e.g., tarceva or erlotinib) or ABL kinase inhibitors (e.g. imatinib). Anti-cancer compounds for co-administration also, include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

The term “co-administration” or “combination therapy” is used to describe a therapy in which at least two active compounds in effective amounts are used to treat cancer or another disease state or condition as otherwise described herein at the same time. Although the term co-administration preferably includes the administration of two active compounds to the patient at the same time, it is not necessary that the compounds be administered to the patient at the same time, although effective amounts of the individual compounds will be present in the patient at the same time.

Co-administered anticancer compounds can include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Co-administration of two or more anticancer agents will often result in a synergistic enhancement of the anticancer activity of the other anticancer agent, an unexpected result. One or more of the present formulations may also be co-administered with another bioactive agent (e.g., antiviral agent, antihyperproliferative disease agent, agents which treat chronic inflammatory disease, among others as otherwise described herein).

The invention therefore enables the development of a gene expression classifier, which may be measured and quantified by gene expression arrays, direct PCR methods to detect quantitative expression of the collection of individual genes that define the signature, or protein based assays that measure the individual quantitative levels of the proteins expressed by the genes in the signature, which can prospectively be used to identify acute leukemia cases which contain mutations or other genetic aberrations that lead to activation of underlying tyrosine kinases. This includes the development of a quantitative algorithm that assesses the expression of the genes/proteins that constitutes this signature to make predictions of response to therapy in ALL patients. The ability to prospectively identify patients with this signature and potential underlying kinase mutations who can be identified and then targeted to therapies incorporating inhibitors or therapeutics targeted to these specific kinase mutations is also a feature of our invention.

Further, as explained above, we provide a method of determining therapeutic outcome in a leukemia patient comprising obtaining tissue from said patient and determining the expression levels of the following genes in said tissue: at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment. In the case of an expectation of unfavorable or unsuccessful treatment, the attending physician will be encouraged to resort to a more aggressive treatment of tyrosine kinase inhibitor therapy and/or alternative therapy.

Alternatively, as explained above, we provide a method of determining therapeutic outcome in a leukemia patient comprising obtaining tissue from said patient and determining the expression levels of the following genes in said tissue: IGJ, CRLF2, MUC4, SPATS2L, SLC2A5, PON2, CA6, NRXN3, DENND3, GPR110, BMPR1B and CD99; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment. In the case of an expectation of unfavorable or unsuccessful treatment, the attending physician will be encouraged to resort to a more aggressive treatment of tyrosine kinase inhibitor therapy and/or alternative therapy.

As explained above, in other embodiments, the present invention provides a method of determining therapeutic outcome in a leukemia patient comprising obtaining tissue from said patient and determining the expression levels of the following genes of said tissue: CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC; and comparing the expression levels of each of said genes from the tissue with a predetermined expression value for each said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

As also explained above, in still other embodiments, our invention provides a method of determining therapeutic outcome in a leukemia patient comprising obtaining tissue from said patient and determining the expression levels of the following genes of said tissue: IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

Our invention provides a method of determining therapeutic outcome in a leukemia patient comprising obtaining tissue from said patient and determining the expression levels of the following genes of said tissue: IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, the present invention provides a method of determining therapeutic outcome in a leukemia patient comprising obtaining tissue from said patient and determining the expression levels of the genes set forth for rankings 1-5 of Table 4 hereof, and optionally, expression levels of one or more genes set forth for rankings 6-21 of Table 4 hereof (at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A), comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, our invention provides a method of determining therapeutic outcome in a leukemia patient comprising obtaining tissue from said patient and determining the expression levels of the genes set forth for rankings 1-19 of Table 2 hereof; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, our invention provides a method of determining therapeutic outcome in a leukemia patient comprising obtaining tissue from said patient and determining the expression levels of the genes set forth for rankings 1-28 of Table 2 hereof; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, our invention provides a method of determining therapeutic outcome in a leukemia patient comprising obtaining tissue from said patient and determining the expression levels of the genes set forth for rankings 1-39 of Table 2 hereof; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, our invention provides a method of determining therapeutic outcome in a leukemia patient comprising obtaining tissue from said patient and determining the expression levels of the genes set forth for rankings 1-64 of Table 2A hereof; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In other embodiments, our invention provides a method of determining therapeutic outcome in a leukemia patient comprising obtaining tissue from said patient and determining the expression levels of the genes set forth for rankings 1-42 of Table 2A hereof; and comparing the expression levels of each of said genes from the tissue with a predetermined expression value for said gene, wherein a level of about the same level or above the predetermined expression value is indicative of an expectation of favorable treatment with tyrosine kinase inhibitor therapy and an expression level below the predetermined expression values is indicative of an expectation of unfavorable or unsuccessful treatment.

In one aspect, the present invention relates to the development of a gene expression classifier, which may be measured and quantified by gene expression arrays, direct PCR methods to detect quantitative expression of the collection of individual genes that define the signature, or protein based assays that measure the individual quantitative levels of the proteins expressed by the genes in the signature, which can prospectively be used to identify acute leukemia cases which contain mutations or other genetic aberrations that lead to activation of underlying tyrosine kinases. This classifier is based upon the gene products and their rankings (relative importance) which are presented in Table 2A and 2B below.

Another aspect of the invention relates to the development of a quantitative algorithm that assesses the expression of the genes/proteins that constitute this signature to make predictions of response to therapy in ALL patients. This algorithm is based upon the gene products and rankings which are presented in Table 2A and 2B below.

A further aspect of the invention relates to the ability to prospectively identify patients with this signature and potential underlying kinase mutations who can be identified and then targeted to therapies incorporating inhibitors or therapeutics targeted to these specific kinase mutations.

Accurate risk stratification constitutes a fundamental paradigm of treatment in acute lymphoblastic leukemia (ALL), allowing the intensity of therapy to be tailored to the patient's therapy, including risk of relapse. The present invention evaluates a gene expression profile related to high risk BCP-ALL and identifies prognostic genes of cancers, in particular leukemia, more particularly high risk B-precursor acute lymphoblastic leukemia, including high risk pediatric acute lymphoblastic leukemia.

Thus, the present invention provides a method of determining the existence of high risk B-precursor ALL in a patient and predicting therapeutic outcome of that patient, especially a pediatric patient. The method comprises the steps of first establishing the threshold value of the genes which appear in Table 2A and 2B and determining whether a patient is a candidate for favorable treatment by a kinase inhibitor, preferably a tyrosine kinase inhibitor, including a JAK or CRLF2 inhibitor, or whether alternative therapy may represent a more favorable approach (i.e. a therapy other than tyrosine kinase inhibitor therapy, including an inhibitor of JAK or CRLF2).

In the present invention, the genes which are presented in Table 2A for Ranks 1-19 (alternatively, the twelve specifically named genes) may be used to predict and/or determine a therapeutic outcome with a tyrosine kinase inhibitor. Preferably, the genes which are presented in Table 2 for Ranks 1-28 are preferably used for the analysis of therapeutic outcome. More preferably, the genes which are presented in Table 2 for Ranks 1-39 and even more preferably, the genes which are presented in Table 2 for Ranks 1-64 are also used for the analysis of therapeutic outcome and a decision as to the use of tyrosine kinase inhibitor therapy (including JAK and/or CRLF2 therapy).

In the present invention, an analysis of the genes which appear in Table 2A or 2B are assessed to determine their level of production in a patient's cancerous tissue and if the genes are expressed at or above a known or predetermined baseline, that patient is a candidate for tyrosine kinase inhibitor therapy, with the prognosis suggesting a favorable outcome (e.g., remission without relapse). If the genes are expressed below a known or predetermined baseline, then the patient is likely not a candidate for tyrosine kinase inhibitor therapy and alternative methods may be counseled. The breakdown of the genes which appear in Table 2, represent those genes which are analyzed according to the present invention to provide a therapeutic prognosis.

Table 2A genes for Ranks 1-19 include the following twelve (12) genes (gene products) which may be analyzed: IGJ, CRLF2, MUC4, SPATS2L, SLC2A5, PON2, CA6, NRXN3, DENND3, GPR110, BMPR1B and CD99.

Table 2A genes for Ranks 1-28 include the following nineteen (19) genes (including the twelve genes from above) which may be readily analyzed: IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC.

Table 2A genes for Ranks 1-39 include the following twenty-five genes (including the nineteen genes from above through Ranks 1-38) which may be readily analyzed in the present invention: IGJ, CRLF2, MUC4, SPATS2L, SLC2A5, PON2, CA6, NRXN3, DENND3, GPRI10, BMPR1B, CD99, SEMA6A, GBP5, IFITMI, TP53INPI, S100Z, ENAM, MDFIC, SCHIP1, RBM47, CHN2, LOC645744, TMEM154 and SLC37A3.

Table 2A genes for Ranks 1-64 include the following 38 genes (including the twenty-five genes from above through Ranks 1-39) for Ranks 1-64 as well as the following nineteen (19) genes: IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157.

The above genes, when over-expressed or expressed at about the same level as a predetermined value, are predictive of a therapeutic outcome using tyrosine kinase inhibitors for therapy of the cancer (remission, successful therapy) of the patient. Thus, the 12 genes from group 1 (Ranks 1-19) of Table 2, when over-expressed or expressed at a predetermined level, are predictive of favorable therapy, as are the 19 genes of group 2 (Ranks 1-28) of Table 2 and the 38 genes of group 3 (Ranks 1-64) of Table 2. The under-expression of these genes is predictive generally of failed therapy with tyrosine kinase inhibitors and provide a rationale for attempting alternative therapy (which may include an increased dosage or different chemotherapeutic protocol, including experimental drug therapy) for the cancer.

In the present invention, the genes which are presented in Table 2B for at least Ranks 1-5 (at least IGJ, SPATS2L, MUC4, CRLF2 and CA6 and optionally, at least one and up to 21 further genes selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A) may be used to predict and/or determine a therapeutic outcome with a tyrosine kinase inhibitor. Preferably, the genes which are presented in Table 4 for Ranks 1-5 and at least one additional gene from ranks 6-26 of Table 4, including Ranks 1-26 ranks of Table 4 are preferably used for the analysis of therapeutic outcome.

According to the present invention, the amount of the prognostic gene(s) (from Table 2A or 2B) from a patient inflicted with high risk B-ALL is determined. The amount of the prognostic gene present in that patient is compared with the established threshold value (a predetermined value) of the prognostic gene(s) which is indicative of therapeutic success (at about the same level or higher than normal/standard expression) or failure (lower than standard/normal expression), whereby the prognostic outcome of the patient for tyrosine kinase inhibitor therapy is determined. The set of prognostic genes may be indicative of a good or favorable prognostic outcome or an unfavorable (bad) outcome. Analyzing expression levels of these genes provides accurate insight (diagnostic and prognostic) information into the likelihood of a therapeutic outcome, especially for tyrosine inhibitor therapy, in ALL, especially in a high risk B-ALL patient, including a pediatric patient.

In certain embodiments, the amount of the prognostic gene(s) is determined by the quantitation of a transcript encoding the sequence of the prognostic gene(s); or a polypeptide encoded by the transcript. The quantitation of the transcript can be based on hybridization to the transcript. The quantitation of the polypeptide can be based on antibody detection or a related method. The method optionally comprises a step of amplifying nucleic acids from the tissue sample before the evaluating (PCR analysis). In a number of embodiments, the evaluating is of a plurality of prognostic genes, preferably at least the five (5) prognostic genes of (ranks 1-5) of Table 4, more preferably at one additional gene from ranks 6-26 of Table 4 and up to 26 genes of Table 4. The prognosis which is determined from measuring the prognostic genes contributes to selection of a therapeutic strategy, which may be tyrosine kinase inhibitor therapy for ALL, including B-precursor ALL (where a favorable prognosis is determined from measurements), or a more aggressive therapy based upon a modification of a traditional therapy or a non-traditional therapy (where an unfavorable prognosis is determined from measurements).

In certain alternative embodiments, the amount of the prognostic gene(s) is determined by the quantitation of a transcript encoding the sequence of the prognostic gene(s); or a polypeptide encoded by the transcript. The quantitation of the transcript can be based on hybridization to the transcript. The quantitation of the polypeptide can be based on antibody detection or a related method. The method optionally comprises a step of amplifying nucleic acids from the tissue sample before the evaluating (PCR analysis). In a number of embodiments, the evaluating is of a plurality of prognostic genes, preferably at least the twelve (12) prognostic genes of group 1 (ranks 1-19) of Table 2A, more preferably at least the 19 genes of group 2 (ranks 1-28) of Table 2A, even more preferably the 38 prognostic genes of group 3 (ranks 1-64) of Table 2A. The prognosis which is determined from measuring the prognostic genes contributes to selection of a therapeutic strategy, which may be tyrosine kinase inhibitor therapy for ALL, including B-precursor ALL (where a favorable prognosis is determined from measurements), or a more aggressive therapy based upon a modification of a traditional therapy or a non-traditional therapy (where an unfavorable prognosis is determined from measurements).

Thus, the present invention is directed to methods for outcome prediction and risk classification in leukemia, especially a high risk classification in B precursor acute lymphoblastic leukemia (ALL), especially in children. In one embodiment, the invention provides a method for classifying the leukemia in a patient that includes obtaining a biological sample from a patient; determining the expression level for the selected group of gene products as presented above, more preferably a group of selected gene products according to those which are set forth in Table 2A or Table 2B hereof, more preferably Table 4 hereof, as described above, to yield an observed gene expression level; and comparing the observed gene expression level for the selected gene products to control gene expression levels (preferably including a predetermined level). The control gene expression level can be the expression level observed for the gene product(s) in a control sample, or a predetermined expression level for the gene product. An observed expression level (at about the same level or higher or lower, depending upon the predetermined value) that is substantially the same as or differs from the control gene expression level is predictive of a therapeutic outcome, in the present invention, for therapy using tyrosine kinase inhibitor(s). In another aspect, the method can include determining a gene expression profile for selected gene products in the biological sample to yield an observed gene expression profile; and comparing the observed gene expression profile for the selected gene products to a control gene expression profile for the selected gene products that correlates with a therapeutic outcome, for example in ALL, and in particular high risk B precursor ALL for therapy with tyrosine kinase inhibitors; wherein a similarity between or higher express levels than the observed gene expression profile and the control gene expression profile is indicative of the potential success for such therapy (e.g., tyrosine kinase inhibitor therapy) and a lower expression level of the observed gene expression profile than the control gene expression profile is indicative of therapeutic failure for tyrosine kinase inhibitor therapy, thereby allowing a decision to try an alternative therapy (i.e., a therapy other than tyrosine kinase inhibition).

The present invention is described in further detail in the examples which follow. It is to be understood that the following is merely exemplary and is not to be taken to limit the scope of the present invention in any way.

### Example 1

### Microarray Modeling

Patient material from 811 cases of pediatric high risk B precursor ALL, from patients derived from Children's Oncology Group (COG) clinical trials P9906 and AALL0232 was run on Affymetrix U133 Plus 2 arrays.^{1,6} RNA was isolated from the diagnostic samples of bone marrow or peripheral blood as previously described. Leukemic blast counts averaged >80% for all cases. The 811 cases were comprised of two cohorts from separate clinical trials: COG P9906 (n=207) and COG AALL0232 (n=604).^{1,6} The RNA was labeled, hybridized to the chips, washed and scanned as previously described. All 811 arrays were normalized together with the RMA algorithm and the default settings for 3′ expression arrays using Affymetrix Expression Console. The simultaneous normalization of all cases was intended to reduce set effects and permit the direct comparison of gene intensities across the different cohorts.

RMA data from the best characterized cases (COG P9906 and the first 283 cases of COG AALL0232) were used as a “training” set to develop the predictive gene expression signature predictive of the Ph-like ALL class, while the remaining 325 cases of COG AALL0232 were defined as the “test set,” to test the performance of the signature. The known kinase mutations and translocations (“events”) for the full cohort of 811 patients are shown in Tables 1A and 1B. Mutation analysis and translocation status for kinase status (except BCR-ABL1, JAK mutations and R8) are currently being completed for the test set. BCR-ABL1 translocations were confirmed by RT-PCR or cytogenetic analysis, with 14 identified in the training set and 21 in the test set. Outlier analysis by recognition of outliers by sampling ends (ROSE)^{5,6} and hierarchical clustering was performed on MASS data for the full set of 811 cases as previously described, identifying 54 cases in the training set with the R8 signature and 49 in the test set. There were an additional 58 kinase events found in the training set: 34 JAK mutations and 25 other events (mutations or translocations; detailed in Table 1B). There was an extensive overlap of many of these features, which resulted in a total of just 89 patients in the test set and 55 in the training set with any kinase event. It is important to note that although the analysis of the training set was comprehensive for the other kinase events (mutations and translocations), this information is not yet fully available for the test set; the actual number of “other kinase” lesions may be larger in the test set when the sequencing and recurrence testing experiments are complete.

TABLE 1A
Known Kinases in Training and Test Sets

| | Training Set | Test Set | Total |
|---|---|---|---|
| | (n = 486) | (n = 325) | (n = 811) |
| BCR-ABL1 | 14 | 21 | 35 |
| R8 | 54 | 49 | 103 |
| JAK mutations | 34 | 13 | 47 |
| other kinases | 25 | NA | 25* |
| Any | 89 | 55* | 144* |

NA = Not available at this time
*indicates actual number may be larger; data still being generated for test set

TABLE 1B
Types of Other Kinases in Training Set

| Type | N |
|---|---|
| IL7 mutation* | 9 |
| EBF1-PDGFRB | 4 |
| NUP214-ABL1 | 3 |
| SH2B3 deletion* | 3 |
| BCR-JAK2 | 1 |
| ETV6-ABL1 | 1 |
| IGH@-EPOR | 1 |
| PAX5-JAK2 | 1 |
| RCSD1-ABL1 | 1 |
| STRN3-JAK2 | 1 |
| Total | 25 |

*one sample had both IL7 mutation and SH2B3 deletion

Using the 89 known tyrosine kinase events, kinase prediction modeling was performed on the training set by the Prediction Analysis of Microarray (PAM) method and three separate optimization criteria: average error, overall error and AUC. Prior to the modeling analysis, 171 probe sets were removed from the dataset (sex-associated, globins and Affymetrix controls) which resulted in a total of 54,504 probe sets being evaluated from the gene expression arrays. The nearest shrunken centroids (NSC) method¹⁶ was used to develop the gene expression models to predict between Ph-like and non-Ph-like ALL cases. The NSC method identifies subsets of discriminating genes through the cross-validation based on certain criterion for prediction accuracy. Three such criteria were used in our study: overall error rate, average of the false positive and false negative rates, and area under the ROC curve (AUC). We performed the NSC analysis using R¹⁷ package pamr.¹⁸ Since pamr only identifies overall error rate, we modified the procedure to also generate the other two criteria. The optimal models based on the three criteria were obtained through the 10-fold 50 repeat cross-validation) performed on the training data set of 486 patients. The accuracies of these optimal models were then estimated through the nested (double loop) cross-validation using the same training data set. The inner loop is the 10 fold 50 repeat cross-validation and the outer loop is the leave one out cross-validation which results in an unbiased internal validation. For external validation we used the optimal models to make predictions on the test data set (n=325) and calculated the error rates based on the predictions. We further examined the association of the Ph-like ALL predictions with event-free survival (EFS) using Kaplan-Meier estimator, Hazard ratio and log-rank (score) test based on the Cox regression.

In our initial evaluation of the gene expression signature we developed for prospective identification of Ph-like ALL cases we included an additional set of cases from the training set in our model building: those B precursor ALL cases which had very high levels of CRLF2 mRNA expression (regardless of the presence or absence of JAK mutations) in addition to the four types of cases selected for modeling as detailed above. We had initially included these ALL cases with high CRLF2 mRNA expression due to the fact that nearly all ALL cases with JAK family kinase mutations were found among high CRLF2-expressing ALL cases.²⁻⁴ At the time of the provisional patent filing, the status of the JAK mutations in the training set was not completely resolved so high CRLF2 mRNA expression was used as a surrogate marker for this genotype. As described below, the optimal models for average error, overall error and AUC from this definition contained 64, 28 and 19 probe sets, respectively. The full list of these 64 probe set, derived from gene expression arrays, is provided in Table 2A.

TABLE 2A
Rank Ordered Probe Set List for the Gene Expression Signature for
Ph-like ALL Cases Derived from Gene Expression Arrays (Including
ALL Cases Expressing high CRLF2 mRNA Levels)

| Rank | Probe set ID | Symbol | Title |
|---|---|---|---|
| 1 | 212592_at | IGJ | immunoglobulin J polypeptide, |
| | | | linker protein for immunoglobulin |
| | | | alpha and mu polypeptides |
| 2 | 208303_s_at | CRLF2 | cytokine receptor-like factor 2 |
| 3 | 217109_at | MUC4 | mucin 4, cell surface associated |
| 4 | 222154_s_at | SPATS2L | spermatogenesis associated, |
| | | | serine-rich 2-like |
| 5 | 204430_s_at | SLC2A5 | solute carrier family 2 (facilitated |
| | | | glucose/fructose transporter), |
| | | | member 5 |
| 6 | 217110_s_at | MUC4 | mucin 4, cell surface associated |
| 7 | 210830_s_at | PON2 | paraoxonase 2 |
| 8 | 201876_at | PON2 | paraoxonase 2 |
| 9 | 206873_at | CA6 | carbonic anhydrase VI |
| 10 | 205795_at | NRXN3 | neurexin 3 |
| 11 | 230161_at | CD99 | CD99 antigen; cluster of |
| | | | differentiation antigen 99; |
| | | | MIC2 or single chain type 1 |
| | | | glycoprotein |
| 12 | 204895_x_at | MUC4 | mucin 4, cell surface associated |
| 13 | 204429_s_at | SLC2A5 | solute carrier family 2 |
| | | | (facilitated glucose/ |
| | | | fructose transporter), member 5 |
| 14 | 242051_at | CD99 | CD99 antigen; cluster of |
| | | | differentiation antigen 99; |
| | | | MIC2 or single chain type 1 |
| | | | glycoprotein |
| 15 | 212975_at | DENND3 | DENN/MADD domain containing 3 |
| 16 | 236489_at | GPR110 | G protein-coupled receptor 110 |
| 17 | 236750_at | NRXN3 | neurexin 3 |
| 18 | 229975_at | BMPR1B | bone morphogenetic protein |
| | | | receptor, type IB |
| 19 | 201028_s_at | CD99 | CD99 antigen; cluster of |
| | | | differentiation antigen 99; |
| | | | MIC2 or single chain type 1 |
| | | | glycoprotein |
| 20 | 225660_at | SEMA6A | scma domain, transmembrane |
| | | | domain (TM), and cytoplasmic |
| | | | domain, (semaphorin) 6A |
| 21 | 229625_at | GBP5 | guanylate binding protein 5 |
| 22 | 214022_s_at | IFITM1 | interferon induced transmembrane |
| | | | protein 1(9-27) |
| 23 | 225912_at | TP53INP1 | tumor protein p53 inducible |
| | | | nuclear protein 1 |
| 24 | 223449_at | SEMA6A | sema domain, transmembrane |
| | | | domain (TM), and cytoplasmic |
| | | | domain, (scmaphorin) 6A |
| 25 | 1554876_a_at | S100Z | S100 calcium binding protein Z |
| 26 | 215028_at | SEMA6A | sema domain, transmembrane |
| | | | domain (TM), and cytoplasmic |
| | | | domain, (semaphorin) 6A |
| 27 | 240586_at | ENAM | Enamelin |
| 28 | 211675_s_at | MDFIC | MyoD family inhibitor |
| | | | domain containing |
| 29 | 201029_s_at | CD99 | CD99 molecule |
| 30 | 201601_x_at | IFITM1 | interferon induced transmembrane |
| | | | protein 1 (9-27) |
| 31 | 242525_at | SLC2A5 | solute carrier family 2 |
| | | | (facilitated glucose/ |
| | | | fructose transporter), member 5 |
| 32 | 238581_at | GBP5 | guanylate binding protein 5 |
| 33 | 204030_s_at | SCHIP1 | schwannomin interacting protein 1 |
| 34 | 218035_s_at | RBM47 | RNA binding motif protein 47 |
| 35 | 235988_at | GPR110 | G protein-coupled receptor 110 |
| 36 | 213385_at | CHN2 | chimerin (chimaerin) 2 |
| 37 | 231241_at | LOC645744 | Similar to PCAF associated |
| | | | factor 65 beta |
| 38 | 238063_at | TMEM154 | transmembrane protein 154 |
| 39 | 223304_at | SLC37A3 | solute carrier family 37 |
| | | | (glycerol-3-phosphate |
| | | | transporter), member 3 |
| 40* | 235112_at | KIAA1958* | — |
| 41 | 212974_at | DENND3 | DENN/MADD domain containing 3 |
| 42 | 215617_at | SPATS2L | spermatogenesis associated, |
| | | | serine-rich 2-like |
| 43 | 223741_s_at | TTYH2 | tweety homolog 2 (Drosophila) |
| 44 | 226002_at | GAB1 | GRB2-associated binding protein 1 |
| 45 | 230643_at | WNT9A | wingless-type MMTV integration |
| | | | site family, member 9A |
| 46 | 242541_at | ABCA9 | ATP-binding cassette, sub-family |
| | | | A (ABC1), member 9 |
| 47 | 239272_at | MMP28 | matrix metallopeptidase 28 |
| 48 | 222496_s_at | RBM47 | RNA binding motif protein 47 |
| 49 | 203372_s_at | SOCS2 | suppressor of cytokine signaling 2 |
| 50 | 229114_at | GAB1 | GRB2-associated binding protein 1 |
| 51 | 218013_x_at | DCTN4 | dynactin 4 (p62) |
| 52 | 222488_s_at | DCTN4 | dynactin 4 (p62) |
| 53 | 1559315_s_at | LOC144481 | hypothetical protein LOC144481 |
| 54 | 225998_at | GAB1 | GRB2-associated binding protein 1 |
| 55 | 238689_at | GPR110 | G protein-coupled receptor 110 |
| 56* | 209524_at | HDGFRP3* | hepatoma-derived growth factor, |
| | | | related protein 3 |
| 57 | 229649_at | NRXN3 | neurexin 3 |
| 58 | 242572_at | GAB1 | GRB2-associated binding protein 1 |
| 59 | 242579_at | BMPR1B | bone morphogenetic protein |
| | | | receptor, type IB |
| 60* | 201334_s_at | ARHGEF12* | Rho guanine nucleotide exchange |
| | | | factor (GEF) 12 |
| 61 | 213371_at | LDB3 | LIM domain binding 3 |
| 62 | 209365_s_at | ECM1 | extracellular matrix protein 1 |
| 63 | 226433_at | RNF157 | ring finger protein 157 |
| 64* | 202388_at | RGS2* | regulator of G-protein |
| | | | signaling 2, 24 kDa |

*Probe sets whose absent or low expression contribute to the signature

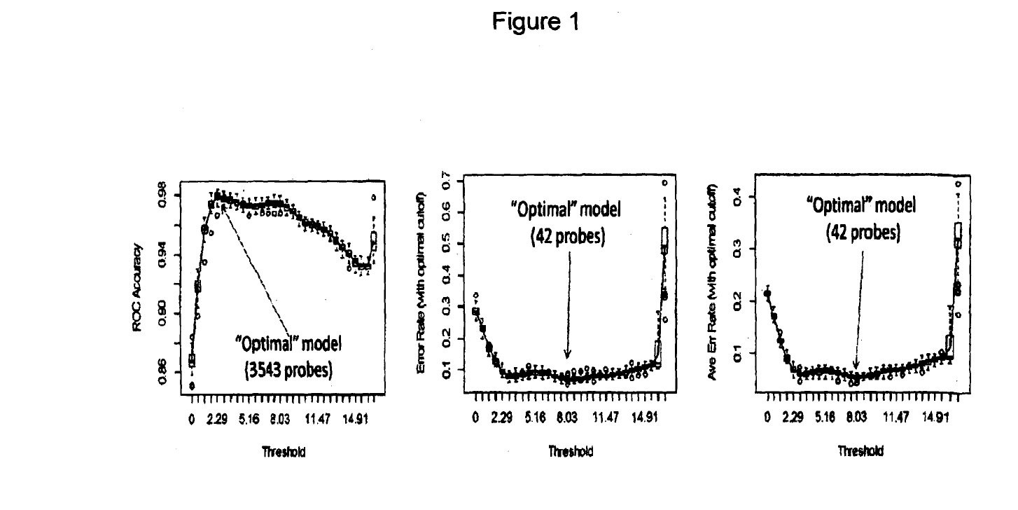

When high CRLF2 expression alone was omitted from the Ph-like criteria, the optimal models for average error, overall error and AUC contained 42, 42 and 3543 probe sets, respectively (FIG. 1). For all three methods, the predicted performance using between 3 and 42 genes was quite similar, with error rates much less than 10% and ROC accuracy >90%. This suggests that most models using between 3 and 42 genes would perform comparably. The full list of these 42 probe sets (corresponding to 26 unique genes) is given in Table 2B, below.

TABLE 2B
Ordered Probe Set List

| Rank | Prob Set ID | Gene Symbol | Gene Title |
|---|---|---|---|
| 1 | 212592_at | IGJ | immunoglobulin J polypeptide, |
| | | | linker protein for immunoglobulin |
| | | | alpha and mu polypeptides |
| 2 | 217109_at | MUC4 | mucin 4, cell surface associated |
| 3 | 222154_s_at | SPATS2L | spermatogenesis associated, |
| | | | serine-rich 2-like |
| 4 | 206873_at | CA6 | carbonic anhydrase VI |
| 5 | 217110_s_at | MUC4 | mucin 4, cell surface associated |
| 6 | 236489_at | GPR110 | G protein-coupled receptor 110 |
| 7 | 210830_s_at | PON2 | paraoxonase 2 |
| 8 | 229975_at | BMPR1B | bone morphogenetic protein |
| | | | receptor, type IB |
| 9 | 201876_at | PON2 | paraoxonase 2 |
| 10 | 204895_x_at | MUC4 | mucin 4, cell surface associated |
| 11 | 208303_s_at | CRLF2 | cytokine receptor-like factor 2 |
| 12 | 205795_at | NRXN3 | neurexin 3 |
| 13 | 204430_s_at | SLC2A5 | solute carrier family 2 |
| | | | (facilitated glucose/ |
| | | | fructose transporter), member 5 |
| 14 | 236750_at | NRXN3 | neurexin 3 |
| 15 | 235988_at | GPR110 | G protein-coupled receptor 110 |
| 16 | 230161_at | CD99 | CD99 molecule |
| 17 | 240586_at_ | ENAM | Enamelin |
| 18 | 214022_s_at | IFITM1 | interferon induced transmembrane |
| | | | protein 1 (9-27) |
| 19 | 201601_x_at | IFITM1 | interferon induced transmembrane |
| | | | protein 1 (9-27) |
| 20 | 225660_at | SEMA6A | sema domain, transmembrane |
| | | | domain (TM), and cytoplasmic |
| | | | domain, (semaphorin) 6A |
| 21 | 223449_at | SEMA6A | sema domain, transmembrane |
| | | | domain (TM), and cytoplasmic |
| | | | domain, (semaphorin) 6A |
| 22 | 238689_at | GPR110 | G protein-coupled receptor 110 |
| 23 | 204429_s_at | SLC2A5 | solute carrier family 2 |
| | | | (facilitated glucose/ |
| | | | fructose transporter), member 5 |
| 24 | 229625_at | GBP5 | guanylate binding protein 5 |
| 25 | 215028_at_ | SEMA6A | sema domain, transmembrane |
| | | | domain (TM), and cytoplasmic |
| | | | domain, (semaphorin) 6A |
| 26 | 213371_at | LDB3 | LIM domain binding 3 |
| 27 | 242051_at | CD99 | CD99 molecule |
| 28 | 211675_s_at | MDFIC | MyoD family inhibitor |
| | | | domain containing |
| 29 | 201028_s_at | CD99 | CD99 molecule |
| 30 | 215617_at | SPATS2L | spermatogenesis associated, |
| | | | serine-rich 2-like |
| 31 | 213385_at | CHN2 | chimerin (chimaerin) 2 |
| 32 | 230643_at | WNT9A | wingless-type MMTV integration |
| | | | site family, member 9A |
| 33 | 225912_at | TP53INP1 | tumor protein p53 inducible |
| | | | nuclear protein 1 |
| 34 | 242579_at | BMPR1B | bone morphogenetic protein |
| | | | receptor, type IB |
| 35 | 223741_s_at | TTYH2 | tweety homolog 2 (Drosophila) |
| 36 | 212975_at | DENND3 | DENN/MADD domain containing 3 |
| 37 | 238063_at | TMEM154 | transmembrane protein 154 |
| 38 | 238581_at | GBP5 | guanylate binding protein 5 |
| 39 | 1554876_a _at | S100Z | S100 calcium binding protein Z |
| 40 | 223304_at | SLC37A3 | solute carrier family 37 |
| | | | (glycerol-3-phosphate |
| | | | transporter), member 3 |
| 41 | 231241_at | LOC645744 | Similar to PCAF associated |
| | | | factor 65 beta |
| 42 | 242525_at | SLC2A5 | solute carrier family 2 |
| | | | (facilitated glucose/ |
| | | | fructose transporter), member 5 |

Receiver operating characteristic analysis (ROC) was applied to the optimization methods to define the cutoff that maximized the true positive rate while minimizing the false positive rate. Using this cutoff (0.278) the performance estimates were evaluated based upon nested (double-loop) cross-validation and prediction in the test set. The results of the cross-validation estimates are shown in Table 3, below. Because of the differences in sample composition between the P9906 and AALL0232 cohorts in the training set, these results are also shown separately. The overall results for the full training set are excellent, and the performance in the subset of AALL0232 patients is slightly better than in P9906. This is particularly important since AALL0232 is more reflective of overall high risk B precursor ALL patients than is P9906.

TABLE 3
Performance Estimates based upon Nested (double-loop) Cross-Validation

| Full Training Set | # probe | | | Error | Average |
|---|---|---|---|---|---|
| Optimization Criterion | sets | Sensitivity | Specificity | rate | error |
| Overall error rate | 42 | 94.4% | 93.7% | 6.2% | 6.0% |
| Average error rate | 42 | 93.3% | 93.5% | 6.6% | 6.6% |
| P9906 subset | # probe | | | Error | Average |
| Optimization Criterion | sets | Sensitivity | Specificity | rate | error |
| Overall error rate | 42 | 91.3% | 97.5% | 3.9% | 5.6% |
| Average error rate | 42 | 89.1% | 97.5% | 4.3% | 6.7% |
| AALL0232 subset | # probe | | | Error | Average |
| Optimization Criterion | sets | Sensitivity | Specificity | rate | error |
| Overall error rate | 42 | 97.7% | 91.1% | 7.9% | 5.6% |
| Average error rate | 42 | 97.7% | 90.7% | 8.2% | 5.8% |

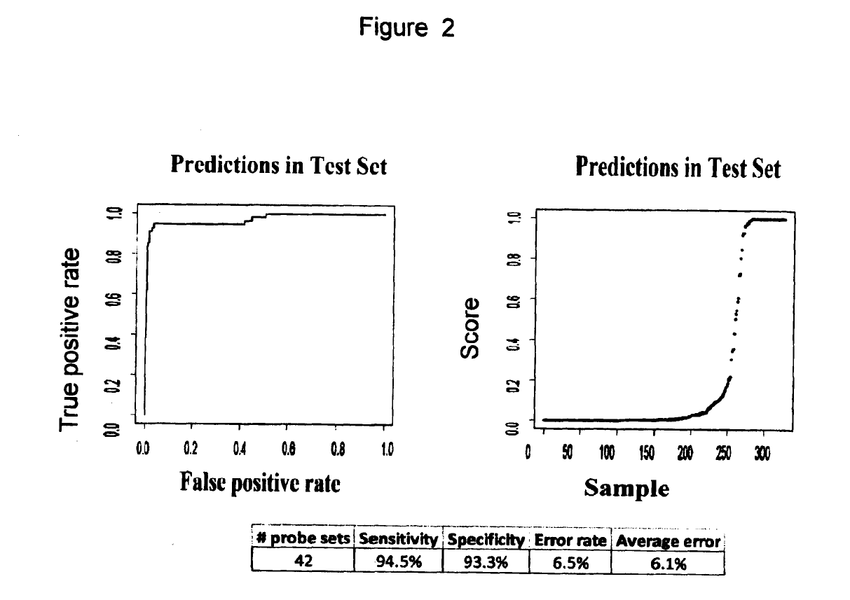

The optimal model of 42 probe sets (Table 2B), and the optimized cutoff value from the ROC analysis in the training set, was applied to the test set to determine its performance. Although only BCR-ABL1, R8 and JAK mutation information was available for the test set, these features accounted for 86.5% (77 of 89) of the known kinase events in the training set (and 100% in the AALL0232 subset of the training set). FIG. 2 shows the performance estimates of this model in the test set. The ROC curve shows excellent predictive power of this model and the intensity plot reflects the clear separation between Ph-like cases and those that are not (cutoff=2.78). Despite the fact that the full extent of kinases in this data set is not known, both the sensitivity and specificity are well over 90%, with error rates around 6%.

### Example 2

### Quantitative PCR Modeling

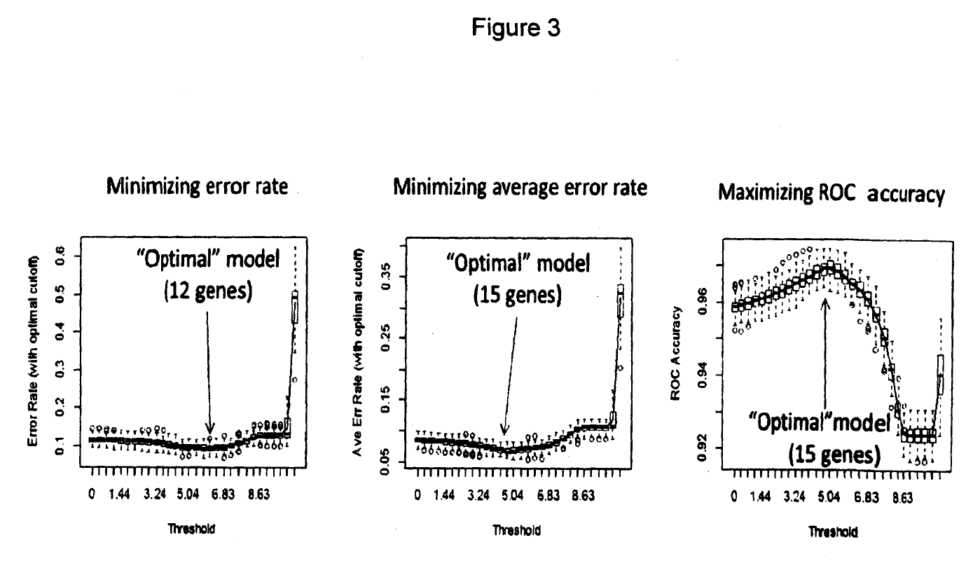

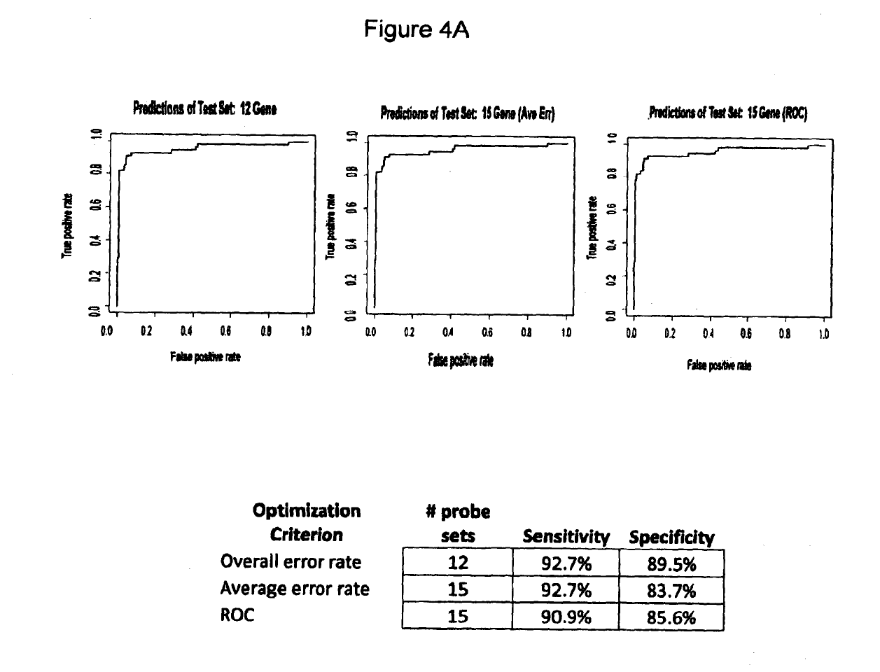

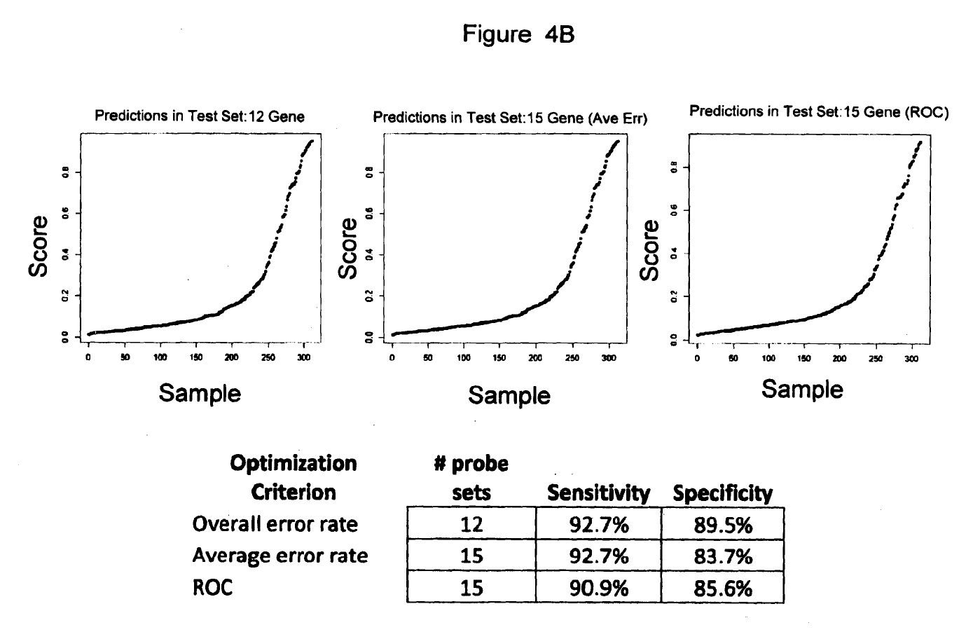

In an effort to demonstrate that this same approach can be applied to a different platform, more amenable to the diagnostic clinical laboratory setting, the same methodologic approach and statistical design was used to develop a model based upon quantitative RT-PCR, rather than gene expression array data. The 42 probe set modeled from the gene expression profiling data (Table 2B) were derived from only 26 unique; as noted in Table 2B some genes were represented by multiple probe sets during the model building. Of these 26 genes, 23 were well characterized and transferrable for evaluation to a direct quantitative RT-PCR assay using the low-density array (LDA) platform of Life Technologies (Table 4). One microgram of RNA was converted to cDNA using random primers and then run using the ABI model 7900ht with default LDA settings outlined by the manufacturer. 478 of the original 486 cases (98.3%) had available material and passed the QC criteria for control gene signal. Optimal gene numbers were determined as was described for the microarray and the results are shown in FIG. 3. Of the 23 genes available on the card, the two best models employed either the top 12 or top 15 genes (Table 4, below). The performance of these models in the test set is shown in FIG. 4. All three models demonstrated sensitivity greater than 90%, although the specificity was just slightly lower. In part, the lower specificity is likely due to the identification of Ph-like cases that are yet to be identified.

TABLE 4
Ordered LDA Gene List

| LDA Rank | Gene | Array Rank |
|---|---|---|
| 1 | IGJ | 1 |
| 2 | SPATS2L | 3, 30 |
| 3 | MUC4 | 2, 4, 10 |
| 4 | CRLF2 | 11 |
| 5 | CA6 | 4 |
| 6 | NRXN3 | 12, 14 |
| 7 | BMPR1B | 8, 34 |
| 8 | GPR110 | 6, 15, 22 |
| 9 | SEMA6A | 20, 21, 25 |
| 10 | PON2 | 7, 9 |
| 11 | CHN2 | 31 |
| 12 | S100Z | 39 |
| 13 | SLC2A5 | 13, 23, 42 |
| 14 | TP53INP1 | 33 |
| 15 | IFITM1 | 18, 19 |
| 16 | GBP5 | 24, 38 |
| 17 | TMEM154 | 37 |
| 18 | CD99 | 16, 27, 29 |
| 19 | MDFIC | 28 |
| 20 | LDB3 | 26 |
| 21 | TTYH2 | 35 |
| 22 | DENND3 | 36 |
| 23 | SLC37A3 | 40 |
| NA | ENAM | 17 |
| NA | LOC645744 | 41 |
| NA | WNT9A | 32 |

Correlation of Models with Outcome

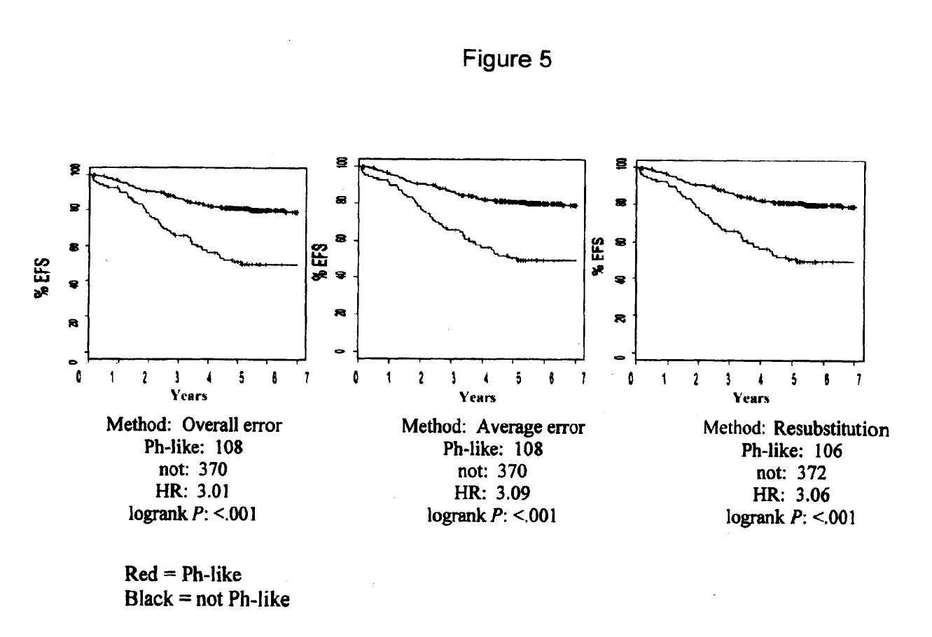

Although the primary focus of this modeling and gene expression signature is the identification of Ph-like ALL cases, our data clearly demonstrate that this gene expression signature is associated with a poor outcome on standard chemotherapy regimens currently employed for ALL therapy. Using the cutoffs determined using the microarray models, FIG. 5 shows the results of modeling with the 42-probe set array data. At present, outcome data are only available for the training set. In addition to the predictions from the two different optimization methods (overall error and average error), a resubstitution plot is also shown. While this is certainly biased, the robustness of the PAM method usually generates results similar to the nested cross-validation. The plots and analysis clearly show the Ph-like ALL cases with significantly inferior outcomes to standard therapies. Within the training set, this held true for the two subsets of cohorts as well.

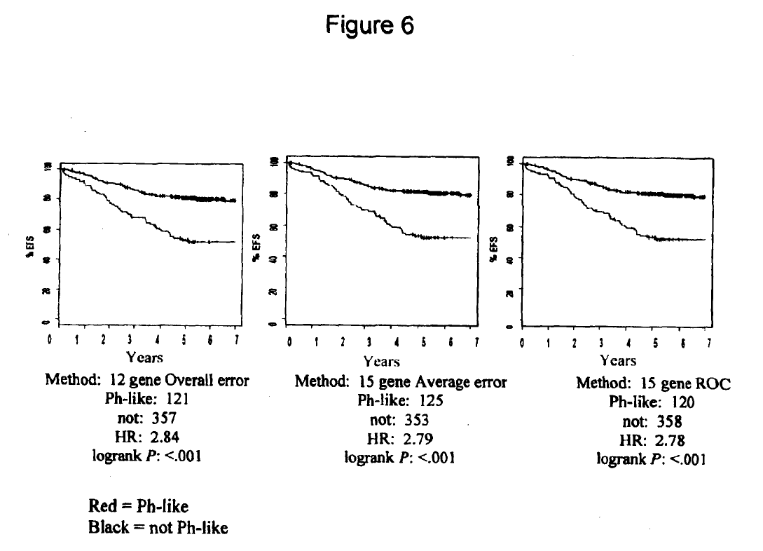

The same analysis was performed using models for 12 and 15 genes derived from the LDA data. These results are shown in FIG. 6. As was true for the microarray models, these models also predicted a group of ALL patients with very poor outcome when treated on standard chemotherapeutic regimens for ALL. Both the hazard ratios and log rank P values were similar to show seen with the microarray data. It should be noted that these models were optimized for detecting the Ph-like ALL patients and not overall outcome. Taken together, however, these data show that patients with this gene expression signature, regardless of whether they have identifiable kinase aberrations, have very poor outcome when treated with standard therapy and may likely benefit from therapy with targeted agents, including tyrosine kinase inhibitor.

### SUMMARY

The tyrosine kinase signature is significantly different than simply genes expressed in BCR-ABL1 cases (something that has been in the literature for several years). High CRLF2 expression, which is very highly correlated with JAK mutations, is rarely seen in cases with BCR-ABL1. This more generalized tyrosine kinase signature identifies a broad spectrum of kinase events (including CRLF2 genomic lesions) and is anticipated to be used to stratify patients into specific targeted therapies. The majority of the cases with this signature have already been shown to have kinase events, however there remain some for whom additional testing is warranted and will likely find similar tyrosine kinase activation mechanisms. While this signature has been defined in pediatric BCP-ALL, it is likely that it will also be present in a subset of adult ALL as well. Finally, the models are very effective at identifying nearly 25% of high risk BCP-ALL cases with significantly worse outcome than the remainder of the cohort. These cases are otherwise indistinguishable and are destined to fail if the current therapeutic regimens are continued. While our focus is toward targeted therapies focused on the kinase pathways, this same testing might be used to stratify patients to identify those who are candidates for alternative therapies.

In terms of platforms, there are not any major limitations. The gene expression patterns for the genes in these models can be identified by any quantitative method for assaying mRNA and, possibly, their protein products (contingent upon the analytical sensitivity of the method). While the optimal models are preferred, it is anticipated that slightly different subsets of these genes and some variation in the menu might give relatively comparable results.

### REFERENCES

1. Kang H, Chen I M, Wilson C S, et al. Gene expression classifiers for relapse-free survival and minimal residual disease improve risk classification and outcome prediction in pediatric B-precursor acute lymphoblastic leukemia. Blood. 2010; 115(7):1394-1405.
2. Chen I M, Harvey R C, Mullighan C G, et al. Outcome modeling with CRLF2, IKZF1, JAK, and minimal residual disease in pediatric acute lymphoblastic leukemia: a Children's Oncology Group study. Blood. 2012; 119(15):3512-3522.
3. Harvey R C, Mullighan C G, Chen I M, et al. Rearrangement of CRLF2 is associated with mutation of JAK kinases, alteration of IKZF1, Hispanic/Latino ethnicity, and a poor outcome in pediatric B-progenitor acute lymphoblastic leukemia. Blood. 2010; 115(26):5312-5321.
4. Mullighan C G, Collins-Underwood J R, Phillips L A, et al. Rearrangement of CRLF2 in B-progenitor- and Down syndrome-associated acute lymphoblastic leukemia. Nat Genet. 2009; 41(11):1243-1246.
5. Harvey R C, Chen I M, Ar K, et al. Identification of Novel Cluster Groups in High-Risk Pediatric B-Precursor Acute Lymphoblastic Leukemia (HR-ALL) by Gene Expression Profiling: Correlation with Clinical and Outcome Variables. a Children's Oncology Group (COG) Study. ASH Annual Meeting Abstracts. 2008; 112(11):2256-.
6. Harvey R C, Mullighan C G, Wang X, et al. Identification of novel cluster groups in pediatric high-risk B-precursor acute lymphoblastic leukemia with gene expression profiling: correlation with genome-wide DNA copy number alterations, clinical characteristics, and outcome. Blood. 2010; 116(23):4874-4884.
7. Loh M L, Zhang J, Harvey R C, et al. Tyrosine kinome sequencing of pediatric acute lymphoblastic leukemia: a report from The Children's Oncology Group TARGET Project. Blood. 2012.
8. Mullighan C G, Su X, Zhang J, et al. Deletion of IKZF1 and prognosis in acute lymphoblastic leukemia. N Engl J Med. 2009; 360(5):470-480.
9. Mullighan C G, Zhang J, Harvey R C, et al. JAK mutations in high-risk childhood acute lymphoblastic leukemia. Proc Natl Acad Sci USA. 2009; 106(23):9414-9418.
10. Roberts K G, Morin R D, Zhang J, et al. Genetic alterations activating kinase and cytokine receptor signaling in high-risk acute lymphoblastic leukemia. Cancer Cell. 2012; 22(2):153-166.
11. Zhang J, Mullighan C G, Harvey R C, et al. Key pathways are frequently mutated in high-risk childhood acute lymphoblastic leukemia: a report from the Children's Oncology Group. Blood. 2011; 118(11):3080-3087.
12. Fielding A K. Current treatment of Philadelphia chromosome-positive acute lymphoblastic leukemia. Hematology Am Soc Hematol Educ Program. 2011; 2011:231-237.
13. Den Boer M L, van Slegtenhorst M, De Menezes R X, et al. A subtype of childhood acute lymphoblastic leukaemia with poor treatment outcome: a genome-wide classification study. Lancet Oncol. 2009; 10(2):125-134.
14. Juric D, Lacayo N J, Ramsey M C, et al. Differential gene expression patterns and interaction networks in BCR-ABL-positive and -negative adult acute lymphoblastic leukemias. Journal of Clinical Oncology. 2007; 25(11):1341-1349.
15. Maude S L, Tasian S K, Vincent T, et al. Targeting JAK1/2 and mTOR in murine xenograft models of Ph-like acute lymphoblastic leukemia. Blood. 2012; 120(17):3510-3518.
16. Tibshirani R, Hastie T, Narasimhan B, Chu G. Diagnosis of multiple cancer types by shrunken centroids of gene expression. Proceedings of the National Academy of Sciences of the United States of America. 2002; 99(10):6567-6572.
17. R Development Core Team. R: A language and environment for statistical computing. Vienna, Austria: R Foundation for Statistical Computing; 2012.
18. Hastie T, Tibshirani R, Narasimhan B, Chu G. pamr: PAM prediction analysis for microarrays; 2011.

1. (canceled)

2. (canceled)

3. (canceled)

4. A method of classifying a subject's B-precursor acute lymphoblastic leukemia (ALL) as being either responsive or non-responsive to tyrosine kinase inhibitor mono or co-therapy, the method comprising:
(a) determining the expression level in a sample obtained from the subject of transcripts or partial transcripts of each member of one or more of a first, second, third or fourth prognostic gene set, thereby deriving an expression pattern profile; and
(b) comparing the expression pattern profile to a reference expression pattern profile;
wherein:
(1) the prognostic gene set consists essentially of at least IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A;
wherein a determination that the sample's expression levels of the gene set is equal to or exceeds its corresponding gene expression reference value indicates that the subject's B-precursor acute lymphoblastic leukemia (ALL) is responsive to tyrosine kinase inhibitor mono or co-therapy.

5. The method of claim 4, wherein derivation of the expression pattern profile and comparison of the expression pattern profile to the reference expression pattern profile involves application of an algorithm to expression level values of the transcripts or partial transcripts of the prognostic gene set.

6. The method of claim 4, wherein a comparison of the expression pattern profile to a reference expression pattern profile which shows an increased level of expression of the transcripts or partial transcripts of the prognostic gene set indicates that the subject's B-precursor acute lymphoblastic leukemia (ALL) is responsive to tyrosine kinase inhibitor monotherapy or cotherapy.

7. The method of claim 4, wherein the step of determining the expression level of the transcripts or partial transcripts of each member of the prognostic gene set involves preparation from the sample of mRNA corresponding to each member of the prognostic gene set.

8. The method of claim 7, wherein the mRNA is amplified by quantitative PCR to produce cDNA.

9. The method of claim 7, wherein the mRNA is amplified by reverse transcription PCR (RT-PCR) to produce cDNA.

10. The method of claim 4, wherein the step of determining the expression level of the transcripts or partial transcripts of each member of the prognostic gene set involves preparation from the sample of polypeptides encoded by each member of prognostic gene set.

11. The method of claim 10, wherein polypeptide expression levels are determined by antibody detection.

12. (canceled)

13. (canceled)

14. (canceled)

15. (canceled)

16. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of a prognostic gene set, thereby deriving an expression pattern profile; and
(b) comparing the expression pattern profile to a reference expression pattern profile;
wherein:
(1) the prognostic gene set is comprised of at least IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A.

17. The method of claim 16, wherein a determination that the expression level of at least one member of the prognostic gene set (preferably all of said members) equals or exceeds its corresponding gene expression control value indicates that the subject's B-precursor acute lymphoblastic leukemia (ALL) is responsive to tyrosine kinase inhibitor mono or co-therapy.

18. The method of claim 16, wherein assaying of the sample comprises gene expression by an array.

19. The method of claim 16, wherein assaying of the sample comprises preparing mRNA from the sample.

20. The method of claim 19, wherein the mRNA is amplified by quantitative PCR to produce cDNA.

21. The method of claim 19, wherein the mRNA is amplified by reverse transcription PCR (RT-PCR) to produce cDNA.

22. The method of claim 4, wherein at least one step of the method is performed in silica.

23. The method of claim 4, wherein the sample is a sample of bone marrow or peripheral blood.

24. The method of claim 4, wherein the reference expression pattern profile is determined by application of an algorithm to control sample expression level values of transcripts or partial transcripts of each member of prognostic gene set.

25. The method of claim 24, wherein the algorithm is generated by kinase prediction modeling of a B-precursor acute lymphoblastic leukemia (ALL) patient training set using the Prediction Analysis of Microarray (PAM) method and the following three separate optimization criteria: average error, overall error and AUC.

26. (canceled)

27. (canceled)

28. (canceled)

29. (canceled)

30. (canceled)

31. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of a prognostic gene set, thereby deriving an expression pattern profile; and
(b) comparing the expression pattern profile to a reference expression pattern profile;
wherein:
(1) the prognostic gene set is comprised of at least IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A; and
(c) determining that the patient's B-precursor acute lymphoblastic leukemia (ALL) will likely be responsive to tyrosine kinase inhibitor mono or co-therapy; and
(d) treating said patient with tyrosine kinase inhibitor mono or co-therapy.

32. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of a prognostic gene set, thereby deriving an expression pattern profile; and
(b) comparing the expression pattern profile to a reference expression pattern profile;
wherein:
(1) the first prognostic gene set is comprised of at least IGJ, SPATS2L, MUC4, CRLF2 and CA6 and optionally, at least one further gene selected from the group consisting of NRXN3; BMPR1B; GPR110; SEMA6A; PON2; CHN2; S100Z; SLC2A5; TP53INP1; IFITM1; GBP5; TMEM154; CD99; MDFIC; LDB3; TTYH2; DENND3; SLC37A3; ENAM; LOC645744 and WNT9A.
(c) determining that the patient's B-precursor acute lymphoblastic leukemia (ALL) will likely not be responsive to tyrosine kinase inhibitor mono or co-therapy; and
(d) treating said patient with anticancer therapy as an alternative to tyrosine kinase inhibitor mono or cotherapy.

33. (canceled)

34. A method of classifying a subject's B-precursor acute lymphoblastic leukemia (ALL) as being either responsive or non-responsive to tyrosine kinase inhibitor mono or co-therapy, the method comprising:
(a) determining the expression level in a sample obtained from the subject of transcripts or partial transcripts of each member of one or more of a first, second, third or fourth prognostic gene set, thereby deriving an expression pattern profile; and
(b) comparing the expression pattern profile to a reference expression pattern profile; wherein:
(1) the first prognostic gene set consists essentially of IGJ, CRLF2, MUC4, SPATS2L, SLC2A5, PON2, CA6, NRXN3, DENND3, GPR110, BMPR1B and CD99;
(2) the second prognostic gene set consists essentially of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC;
(3) the third prognostic gene consists essentially of IGJ CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and
(4) the fourth prognostic gene consists essentially of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157;
wherein a determination that the sample's expression levels of at least one member of the first, second, third or fourth gene sets is equal to or exceeds its corresponding gene expression reference value indicates that the subject's B-precursor acute lymphoblastic leukemia (ALL) is responsive to tyrosine kinase inhibitor mono or co-therapy.

35. (canceled)

36. (canceled)

37. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of one or more of a first, second, third or fourth prognostic gene set, thereby deriving an expression pattern profile; and
(b) comparing the expression pattern profile to a reference expression pattern profile;
wherein:
(1) the first prognostic gene set is comprised of IGJ, CRLF2, MUC4, SPATS2L, SLC2A5, PON2, CA6, NRXN3, DENND3, GPR110, BMPR1B and CD99;
(2) the second prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z ENAM, and MDFIC;
(3) the third prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and
(4) the fourth prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154, SLC37,43, TTYH2, GAB1, WNT9A, ABCA9, MMP28, SOC2S, DCTN4, LOC14481, HDGFRP3, ARHGEF12, LDB3, ECM1 and RNF157.

38. (canceled)

39. (canceled)

40. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of one or more of a first, second, third or fourth prognostic gene set, thereby deriving an expression pattern profile; and
(b) comparing the expression pattern profile to a reference expression pattern profile;
wherein:
(1) the first prognostic gene set is comprised of IGJ, CRLF2, MUC4, SPATS2L, SLC2A5, PON2, CA6, NRXN3, DENND3, GPR110, BMPR1B and CD99;
(2) the second prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC;
(3) the third prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and
(4) the fourth prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157; and
(e) determining that the patient's B-precursor acute lymphoblastic leukemia (ALL) will likely be responsive to tyrosine kinase inhibitor mono or co-therapy; and
(f) treating said patient with tyrosine kinase inhibitor mono or co-therapy.

41. A method of determining whether a subject's B-precursor acute lymphoblastic leukemia (ALL) is responsive to tyrosine kinase inhibitor mono or co-therapy, the method comprising:
(a) assaying a sample obtained from the subject to determine the expression level of transcripts or partial transcripts of each member of one or more of a first, second, third or fourth prognostic gene set, thereby deriving an expression pattern profile; and
(b) comparing the expression pattern profile to a reference expression pattern profile;
wherein:
(1) the first prognostic gene set is comprised of IGJ, CRLF2, MUC4, SPATS2L, SLC2A5, PON2, CA6, NRXN3, DENND3, GPR110, BMPR1B and CD99;
(2) the second prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, and MDFIC;
(3) the third prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z, ENAM, MDFIC, SCHIP1, RBM47, CHN2, LOC645744, TMEM154 and SLC37A3; and
(4) the fourth prognostic gene set is comprised of IGJ, CRLF2, MUC4, SPATS2L, SLC2A5, PON2, CA6, NRXN3, DENND3, GPR110, BMPR1B, CD99, SEMA6A, GBP5, IFITMI, TP53INPI, S100Z ENAM, MDFIC, SCHIP1, RBM47, CHN2, LOC645744, TMEM154, SLC37A3, TTYH2, GAB1, WNT9A, ABCA9, MMP28, SOC2S, DCTN4, LOC14481, HDGFRP3, ARHGEF12, LDB3, ECM1 and RNF157; and
(e) determining that the patient's B-precursor acute lymphoblastic leukemia (ALL) will likely not be responsive to tyrosine kinase inhibitor mono or co-therapy; and
(f) treating said patient with anticancer therapy as an alternative to tyrosine kinase inhibitor mono or cotherapy.
